SEQUENCE LISTING (189 sequences)

SEQ ID NO: 1 - DNA, 22 bp, Artificial sequence
  Synthetic oligonucleotide
ggaagatgtaggtagggatcga

SEQ ID NO: 2 - DNA, 22 bp, Artificial sequence
  Synthetic oligonucleotide
tcatgttggtcactgccgtgtg

SEQ ID NO: 3 - DNA, 22 bp, Artificial sequence
  Synthetic oligonucleotide
ttagtgtatgtagcatgcgaca

SEQ ID NO: 4 - DNA, 22 bp, Artificial sequence
  Synthetic oligonucleotide
tgtatgtagcagagtgggtatg

SEQ ID NO: 5 - DNA, 22 bp, Artificial sequence
  Synthetic oligonucleotide
agaggatgtagtggccccgccg

SEQ ID NO: 6 - DNA, 22 bp, Artificial sequence
  Synthetic oligonucleotide
gatcgggaagtagatatggcgc

SEQ ID NO: 7 - DNA, 22 bp, Artificial sequence
  Synthetic oligonucleotide
gagacaggtagaagggcccagg

SEQ ID NO: 8 - DNA, 22 bp, Artificial sequence
  Synthetic oligonucleotide
tggagtggatcagtgcgcactg

SEQ ID NO: 9 - DNA, 22 bp, Artificial sequence
  Synthetic oligonucleotide
tggataacgcttgtgcaggctg

SEQ ID NO: 10 - DNA, 22 bp, Artificial sequence
  Synthetic oligonucleotide
cgaaccattcacagcacaaaca

SEQ ID NO: 11 - DNA, 22 bp, Artificial sequence
  Synthetic oligonucleotide
ttcaacaggtcttgtgtggcca

SEQ ID NO: 12 - DNA, 22 bp, Artificial sequence
  Synthetic oligonucleotide
tggtgcagttgtagatcccccg

SEQ ID NO: 13 - DNA, 22 bp, Artificial sequence
  Synthetic oligonucleotide
tcaccatggaacacacggcccg

SEQ ID NO: 14 - DNA, 22 bp, Artificial sequence
  Synthetic oligonucleotide
ctacacgcctggtccacgtgcc

SEQ ID NO: 15 - DNA, 22 bp, Artificial sequence
  Synthetic oligonucleotide
ctacacgtgcccggtggacatg

SEQ ID NO: 16 - DNA, 22 bp, Artificial sequence
  Synthetic oligonucleotide
ctcctagtgccgctcccgtgcc

SEQ ID NO: 17 - DNA, 22 bp, Artificial sequence
  Synthetic oligonucleotide
gtgtgggggacacattgtgaca

SEQ ID NO: 18 - DNA, 22 bp, Artificial sequence
  Synthetic oligonucleotide
tcaccaatgttgcatgcgcacg

SEQ ID NO: 19 - DNA, 22 bp, Artificial sequence
  Synthetic oligonucleotide
ttccacatcactttgcttggca

SEQ ID NO: 20 - DNA, 22 bp, Artificial sequence
  Synthetic oligonucleotide
gtagacggagaggtccttgtgt

SEQ ID NO: 21 - DNA, 22 bp, Artificial sequence
  Synthetic oligonucleotide
gcacttggaacaggatcgaccg

SEQ ID NO: 22 - DNA, 22 bp, Artificial sequence
  Synthetic oligonucleotide
gggaaaccgctaagcagtggca

SEQ ID NO: 23 - DNA, 22 bp, Artificial sequence
  Synthetic oligonucleotide
cgagccagaggaaggtgtgccc

SEQ ID NO: 24 - DNA, 23 bp, Artificial sequence
  Synthetic oligonucleotide
gttgggaaagaggcatcagctgc

SEQ ID NO: 25 - DNA, 21 bp, Artificial sequence
  Synthetic oligonucleotide
tactgcgggatgctgctccac

SEQ ID NO: 26 - DNA, 22 bp, Artificial sequence
  Synthetic oligonucleotide
ctgagttgtacacatctggacg

SEQ ID NO: 27 - DNA, 22 bp, Artificial sequence
  Synthetic oligonucleotide
cttaactgttcatgctcgtgca

SEQ ID NO: 28 - DNA, 22 bp, Artificial sequence
  Synthetic oligonucleotide
tcaacatcttccttagtcccag

SEQ ID NO: 29 - DNA, 22 bp, Artificial sequence
  Synthetic oligonucleotide
gtccacacgtctggcgccgtcc

SEQ ID NO: 30 - DNA, 22 bp, Artificial sequence
  Synthetic oligonucleotide
ctatgtatggcgagttggcgca

SEQ ID NO: 31 - DNA, 22 bp, Artificial sequence
  Synthetic oligonucleotide
cggagtgtgtcgtcctagccca

SEQ ID NO: 32 - DNA, 22 bp, Artificial sequence
  Synthetic oligonucleotide
ccctcaaaatagtgtttgccca

SEQ ID NO: 33 - DNA, 22 bp, Artificial sequence
  Synthetic oligonucleotide
atggcgaagggaacgggcagca

SEQ ID NO: 34 - DNA, 22 bp, Artificial sequence
  Synthetic oligonucleotide
tagcctctgatcctcgtccaaa

SEQ ID NO: 35 - DNA, 22 bp, Artificial sequence
  Synthetic oligonucleotide
tggtacagctgttcagtctaca

SEQ ID NO: 36 - DNA, 22 bp, Artificial sequence
  Synthetic oligonucleotide
ggtttatcaaagtggcaatgca

SEQ ID NO: 37 - DNA, 22 bp, Artificial sequence
  Synthetic oligonucleotide
ccgggattcgtgatgtggcgtg

SEQ ID NO: 38 - DNA, 22 bp, Artificial sequence
  Synthetic oligonucleotide
gcgaagataaagaggacgtgca

SEQ ID NO: 39 - DNA, 22 bp, Artificial sequence
  Synthetic oligonucleotide
ttagagttggcatcaccatcgg

SEQ ID NO: 40 - DNA, 23 bp, Artificial sequence
  Synthetic oligonucleotide
agtaggagaaggcggtgcgtccc

SEQ ID NO: 41 - DNA, 22 bp, Artificial sequence
  Synthetic oligonucleotide
agattgagtacagccatgcgga

SEQ ID NO: 42 - DNA, 22 bp, Artificial sequence
  Synthetic oligonucleotide
ggcccaatgatgaatacacagc

SEQ ID NO: 43 - DNA, 22 bp, Artificial sequence
  Synthetic oligonucleotide
ttatgattcgggagctgatacg

SEQ ID NO: 44 - DNA, 65 bp, Artificial sequence
  Synthetic oligonucleotide
gcacagggccacaataatggcatttgttggggtaaccaactatttgttactgttgttgatactac

SEQ ID NO: 45 - DNA, 65 bp, Artificial sequence
  Synthetic oligonucleotide
gcacagggccacaataatggtatttgttggggtaatcaactgtttgttactgttgttgatactac

SEQ ID NO: 46 - DNA, 65 bp, Artificial sequence
  Synthetic oligonucleotide
gcacagggccacaataatggtatttgctggggaaaccacttgtttgttactgttgttgatactac

SEQ ID NO: 47 - DNA, 65 bp, Artificial sequence
  Synthetic oligonucleotide
gcacagggccacaataatggtgtttgctggcataatcaattatttgttactgttgttgatactac

SEQ ID NO: 48 - DNA, 65 bp, Artificial sequence
  Synthetic oligonucleotide
gcacagggccacaataatggtatttgttggggcaatcagttatttgttactgttgttgatactac

SEQ ID NO: 49 - DNA, 65 bp, Artificial sequence
  Synthetic oligonucleotide
gcacagggccacaataatggtatttgttggggcaatcaggtatttgttactgttgttgatactac

SEQ ID NO: 50 - DNA, 20 bp, Artificial sequence
  Synthetic oligonucleotide
gcacagggccacaataatgg

SEQ ID NO: 51 - DNA, 23 bp, Artificial sequence
  Synthetic oligonucleotide
gtagtatcaacaacagtaacaaa

SEQ ID NO: 52 - DNA, 70 bp, Artificial sequence
  Synthetic oligonucleotide
gcctgttgtgagcctcctgtcgaannnnnnnnnnnnnnnnnnnnnnttgagcgtttattcttgtctccca

SEQ ID NO: 53 - DNA, 24 bp, Artificial sequence
  Synthetic oligonucleotide
ttcgacaggaggctcacaacaggc

SEQ ID NO: 54 - DNA, 24 bp, Artificial sequence
  Synthetic oligonucleotide
tgggagacaagaataaacgctcaa

SEQ ID NO: 55 - DNA, 24 bp, Artificial sequence
  Synthetic oligonucleotide
gcctgttgtgagcctcctgtcgaa

SEQ ID NO: 56 - DNA, 23 bp, Artificial sequence
  Synthetic oligonucleotide
gtdgayachachmgnagyachaa

SEQ ID NO: 57 - DNA, 25 bp, Artificial sequence
  Synthetic oligonucleotide
gaaaaataaactgtaaatcatattc

SEQ ID NO: 58 - DNA, 25 bp, Artificial sequence
  Synthetic oligonucleotide
gaaaaataaactgtaaatcatactc

SEQ ID NO: 59 - DNA, 25 bp, Artificial sequence
  Synthetic oligonucleotide
gaaaaataaactgtaaatcaaattc

SEQ ID NO: 60 - DNA, 25 bp, Artificial sequence
  Synthetic oligonucleotide
gaaaaataaactgtaaatcaaactc

SEQ ID NO: 61 - DNA, 91 bp, Artificial sequence
  Synthetic oligonucleotide
acgcagtaccaacatgacattatgtgcatccgtaactacatcttccacatacaccaattctgattataaagagtacatgcgtcatgtggaa

SEQ ID NO: 62 - DNA, 91 bp, Artificial sequence
  Synthetic oligonucleotide
acgcagtacaaatatgacactatgtgcatctgtgtctaaatctgctacatacactaattcagattataaggaatacatgcgccatgtggag

SEQ ID NO: 63 - DNA, 97 bp, Artificial sequence
  Synthetic oligonucleotide
acgcagtactaacatgactgtgtgtgcagccactacatcatctctttcagacacatataaggccacagaatataaacagtacatgcgacatgtagaa

SEQ ID NO: 64 - DNA, 94 bp, Artificial sequence
  Synthetic oligonucleotide
acgcagtacaaatatgtcattatgtgctgccatatctacttcagaaactacatataaaaatactaactttaaggagtacctacgacatggggag

SEQ ID NO: 65 - DNA, 97 bp, Artificial sequence
  Synthetic oligonucleotide
tcccagtaccaatttaacaatatgtgcttctacacagtctcctgtacctgggcaatatgatgctaccaaatttaagcagtatagcagacatgttgag

SEQ ID NO: 66 - DNA, 97 bp, Artificial sequence
  Synthetic oligonucleotide
ccgcagtactaaccttaccattagtacattatctgcagcatctgcatccactccatttaaaccatctgattataaacaatttataagacatggcgaa

SEQ ID NO: 67 - DNA, 91 bp, Artificial sequence
  Synthetic oligonucleotide
taggaacacaaacatgactatatctgcaaccacacaaacgttatccacatataattcaagccaaattaaacagtatgtaagacatgtagag

SEQ ID NO: 68 - DNA, 94 bp, Artificial sequence
  Synthetic oligonucleotide
acgtagtaccaatatgtctgtttgtgctgcaattgcaaacagtgatactacatttaaaagtagtaattttaaagagtatttaagacatggtgag

SEQ ID NO: 69 - DNA, 91 bp, Artificial sequence
  Synthetic oligonucleotide
tcgcagtactaatatgactttatgcacacaagtaactagtgacagtacatataaaaatgaaaattttaaagaatatataagacatgttgaa

SEQ ID NO: 70 - DNA, 100 bp, Artificial sequence
  Synthetic oligonucleotide
tagaagcacaaacttttcagtttgtgtaggtacacaatccacaagtacaactgcaccatatgcaaacagtaattttaaggaatacctcagacatgcagaa

SEQ ID NO: 71 - DNA, 94 bp, Artificial sequence
  Synthetic oligonucleotide
ccgtagtacaaatatgtctgtgtgttctgctgtgtcttctagtgacagtacatataaaaatgacaattttaaggaatatttaaggcatggtgaa

SEQ ID NO: 72 - DNA, 97 bp, Artificial sequence
  Synthetic oligonucleotide
ccgtagtaccaactttacattatctacctctatagagtcttccataccttctacatatgatccttctaagtttaaggaatataccaggcacgtggag

SEQ ID NO: 73 - DNA, 97 bp, Artificial sequence
  Synthetic oligonucleotide
tcgtagcactaatttaaccttatgtgctgccacacagtcccccacaccaaccccatataataacagtaatttcaaggaatatttgcgtcatggggag

SEQ ID NO: 74 - DNA, 91 bp, Artificial sequence
  Synthetic oligonucleotide
ccgtagtactaacatgactttgtgtgccactgcaacatctggtgatacatatacagctgctaattttaaggaatatttaagacatgctgaa

SEQ ID NO: 75 - DNA, 97 bp, Artificial sequence
  Synthetic oligonucleotide
tcgtagtacaaacttgacgttatgtgcctctactgaccctactgtgcccagtacatatgacaatgcaaagtttaaggaatacttgcggcatgtggaa

SEQ ID NO: 76 - DNA, 97 bp, Artificial sequence
  Synthetic oligonucleotide
ccgtagtacaaacatgacaatatgtgctgccactacacagtcccctccgtctacatatactagtgaacaatataagcaatacatgcgacatgttgag

SEQ ID NO: 77 - DNA, 97 bp, Artificial sequence
  Synthetic oligonucleotide
ccgcagtactaatttaacattatgtgcctctacacaaaatcctgtgccaagtacatatgaccctactaagtttaagcagtatagtagacatgtggag

SEQ ID NO: 78 - DNA, 94 bp, Artificial sequence
  Synthetic oligonucleotide
cagaagtacaaatttaactattagcactgccactgctgcggtttccccaacatttactccaagtaactttaagcaatatattaggcatggggaa

SEQ ID NO: 79 - DNA, 91 bp, Artificial sequence
  Synthetic oligonucleotide
tcgtagcactaacatgactttatgtgctgaggttaaaaaggaaagcacatataaaaatgaaaattttaaggaataccttcgtcatggcgag

SEQ ID NO: 80 - DNA, 91 bp, Artificial sequence
  Synthetic oligonucleotide
caggaatacaaacatgactctttccgcaaccacacagtctatgtctacatataattcaaagcaaattaaacagtatgttagacatgcagag

SEQ ID NO: 81 - DNA, 91 bp, Artificial sequence
  Synthetic oligonucleotide
ccgtagtactaacctaacattgtgtgctacagcatccacgcaggatagctttaataattctgactttagggagtatattagacatgtggag

SEQ ID NO: 82 - DNA, 97 bp, Artificial sequence
  Synthetic oligonucleotide
acgtagtacaaacatgacaatatgtgctgctacaactcagtctccatctacaacatataatagtacagaatataaacaatacatgcgacatgttgag

SEQ ID NO: 83 - DNA, 91 bp, Artificial sequence
  Synthetic oligonucleotide
tagaagtactaacatgactattagtactgctacagaacagttaagtaaatatgatgcacgaaaaattaatcagtaccttagacatgtggag

SEQ ID NO: 84 - DNA, 91 bp, Artificial sequence
  Synthetic oligonucleotide
tcgtagcactaatatgacattatgcactgaagtaactaaggaaggtacatataaaaatgataattttaaggaatatgtacgtcatgttgaa

SEQ ID NO: 85 - DNA, 97 bp, Artificial sequence
  Synthetic oligonucleotide
tcgcagcaccaatctttctgtgtgtgcttctactacttcttctattcctaatgtatacacacctaccagttttaaagaatatgccagacatgtggag

SEQ ID NO: 86 - DNA, 94 bp, Artificial sequence
  Synthetic oligonucleotide
ccgcagtactaatttaaccatttgtactgctacatccccccctgtatctgaatataaagccacaagctttagggaatatttgcgccatacagag

SEQ ID NO: 87 - DNA, 91 bp, Artificial sequence
  Synthetic oligonucleotide
cagaagtactaattttactatttgtaccgcctccactgctgcagcagaatacacggctaccaactttagggaatttttgcgacacacggag

SEQ ID NO: 88 - DNA, 100 bp, Artificial sequence
  Synthetic oligonucleotide
cagaagtacaaacttttctgtttgtgtaggcacacaatccacaagtacaaatccaccatatgcaaacactaattttaaggaatacctaaggcatgcagaa

SEQ ID NO: 89 - DNA, 91 bp, Artificial sequence
  Synthetic oligonucleotide
cagaagcaccaacatgactattaatgcagctaaaagcacattaactaaatatgatgcccgtgaaatcaatcaataccttcgccatgtggag

SEQ ID NO: 90 - DNA, 91 bp, Artificial sequence
  Synthetic oligonucleotide
acgtagtaccaacatgactttatgttctgaggaaaaatcagaggctacatacaaaaatgaaaactttaaggaataccttagacatgtggaa

SEQ ID NO: 91 - DNA, 97 bp, Artificial sequence
  Synthetic oligonucleotide
tcgcagtaccaattttactttgtctactactactgaatcagctgtaccaaatatttatgatcctaataaatttaaggaatatattaggcatgttgag

SEQ ID NO: 92 - DNA, 97 bp, Artificial sequence
  Synthetic oligonucleotide
ccgcagtaccaacctcactattagtactgtatctgcacaatctgcatctgccacttttaaaccatcagattataagcagtttataaggcatggtgag

SEQ ID NO: 93 - DNA, 91 bp, Artificial sequence
  Synthetic oligonucleotide
ccgtagcaccaatattactatttctgcagccacatcacagtccggtgaataccaggcctctaactttaaggaatacctacgccacacagaa

SEQ ID NO: 94 - DNA, 94 bp, Artificial sequence
  Synthetic oligonucleotide
tcgcagtactaatgtaactatttgtactgccacagcgtcctctgtatcagaatatacagcttctaattttcgtgagtatcttcgccacactgag

SEQ ID NO: 95 - DNA, 100 bp, Artificial sequence
  Synthetic oligonucleotide
tagaagcactaatttttctgtatgtgtaggtacacaggctagtagctctactacaacgtatgccaactctaattttaaggaatatttaagacatgcagaa

SEQ ID NO: 96 - DNA, 97 bp, Artificial sequence
  Synthetic oligonucleotide
acgcagtactaacatgactgtgtgtgctcctacctcacaatcgccttctgctacatataatagttcagactacaaacaatacatgcgacatgtggag

SEQ ID NO: 97 - DNA, 97 bp, Artificial sequence
  Synthetic oligonucleotide
tagaagtaccaatttaaccattagcactgctgttactcaatctgttgcacaaacatttactccagcaaactttaagcaatacattaggcatggggaa

SEQ ID NO: 98 - DNA, 97 bp, Artificial sequence
  Synthetic oligonucleotide
acgtagtactaattttacattgtctgcctgcaccgaaacggccatacctgctgtatatagccctacaaagtttaaggaatatactaggcatgtggag

SEQ ID NO: 99 - DNA, 91 bp, Artificial sequence
  Synthetic oligonucleotide
ccgcagcaccaattttactattagtgctgctaccaacaccgaatcagaatataaacctaccaattttaaggaatacctaagacatgtggag

SEQ ID NO: 100 - DNA, 23 bp, Artificial sequence
  Synthetic oligonucleotide
agtagttcaaactgttgattacc

SEQ ID NO: 101 - DNA, 11 bp, Artificial sequence
  Synthetic oligonucleotide
gtttccccagc

SEQ ID NO: 102 - DNA, 14 bp, Artificial sequence
  Synthetic oligonucleotide
cagtacaaatagtt

SEQ ID NO: 103 - DNA, 13 bp, Artificial sequence
  Synthetic oligonucleotide
tgccagcgaacac

SEQ ID NO: 104 - DNA, 22 bp, Artificial sequence
  Synthetic oligonucleotide
taactgattgccctttggttct

SEQ ID NO: 105 - DNA, 13 bp, Artificial sequence
  Synthetic oligonucleotide
aagacctgttgaa

SEQ ID NO: 106 - DNA, 13 bp, Artificial sequence
  Synthetic oligonucleotide
aagacatgttgaa

SEQ ID NO: 107 - DNA, 13 bp, Artificial sequence
  Synthetic oligonucleotide
aagacatgtggag

SEQ ID NO: 108 - DNA, 13 bp, Artificial sequence
  Synthetic oligonucleotide
aagacatgtagag

SEQ ID NO: 109 - DNA, 13 bp, Artificial sequence
  Synthetic oligonucleotide
cagacatgttgag

SEQ ID NO: 110 - DNA, 13 bp, Artificial sequence
  Synthetic oligonucleotide
cagacatgtggag

SEQ ID NO: 111 - DNA, 13 bp, Artificial sequence
  Synthetic oligonucleotide
aagacatggcgaa

SEQ ID NO: 112 - DNA, 13 bp, Artificial sequence
  Synthetic oligonucleotide
aaggcatggtgaa

SEQ ID NO: 113 - DNA, 13 bp, Artificial sequence
  Synthetic oligonucleotide
aaggcatggtgag

SEQ ID NO: 114 - DNA, 13 bp, Artificial sequence
  Synthetic oligonucleotide
aagacatggtgag

SEQ ID NO: 115 - DNA, 13 bp, Artificial sequence
  Synthetic oligonucleotide
acgtcatgttgaa

SEQ ID NO: 116 - DNA, 21 bp, Artificial sequence
  Synthetic oligonucleotide
cagttacaaatagttggttac

SEQ ID NO: 117 - DNA, 64 bp, Artificial sequence
  Synthetic oligonucleotide
cacagggccacaataatggcatttgttggggtaaccaactatttgttactgttgttgatactac

SEQ ID NO: 118 - DNA, 64 bp, Artificial sequence
  Synthetic oligonucleotide
cacagggccacaataatggtatttgttggggtaatcaactgtttgttactgttgttgatactac

SEQ ID NO: 119 - DNA, 64 bp, Artificial sequence
  Synthetic oligonucleotide
cacagggccacaataatggtatttgctggggaaaccacttgtttgttactgttgttgatactac

SEQ ID NO: 120 - DNA, 64 bp, Artificial sequence
  Synthetic oligonucleotide
cacagggccacaataatggtatatgttggggcaatcacttgtttgttactgttgttgatactac

SEQ ID NO: 121 - DNA, 64 bp, Artificial sequence
  Synthetic oligonucleotide
cacagggccacaataatggtatctgttggggcaatcaattgtttgttactgttgttgatactac

SEQ ID NO: 122 - DNA, 64 bp, Artificial sequence
  Synthetic oligonucleotide
cacagggccacaataatggcatttgttggggcaaccaggtatttgttactgttgttgatactac

SEQ ID NO: 123 - DNA, 64 bp, Artificial sequence
  Synthetic oligonucleotide
cacagggccacaataatggcatttgctggcataatcaactgtttgttactgttgttgatactac

SEQ ID NO: 124 - DNA, 64 bp, Artificial sequence
  Synthetic oligonucleotide
cacagggccacaataatggcatatgttttggcaatcagttatttgttactgttgttgatactac

SEQ ID NO: 125 - DNA, 64 bp, Artificial sequence
  Synthetic oligonucleotide
cacagggccacaataatggtatatgttggggaaatcagctatttgttactgttgttgatactac

SEQ ID NO: 126 - DNA, 64 bp, Artificial sequence
  Synthetic oligonucleotide
cacagggccacaataatggcatttgttttgggaatcagttgtttgttactgttgttgatactac

SEQ ID NO: 127 - DNA, 64 bp, Artificial sequence
  Synthetic oligonucleotide
cacagggccacaataatggtatttgttggggaaatcagttatttgttactgttgttgatactac

SEQ ID NO: 128 - DNA, 64 bp, Artificial sequence
  Synthetic oligonucleotide
cacagggccacaataatggcatctgttggaacaatcagttatttgttactgttgttgatactac

SEQ ID NO: 129 - DNA, 64 bp, Artificial sequence
  Synthetic oligonucleotide
cacagggccacaataatggtatttgttggggcaatcaggtgtttgttactgttgttgatactac

SEQ ID NO: 130 - DNA, 64 bp, Artificial sequence
  Synthetic oligonucleotide
cacagggccacaataatggtatttgttgggggaatcagttatttgttactgttgttgatactac

SEQ ID NO: 131 - DNA, 64 bp, Artificial sequence
  Synthetic oligonucleotide
cacagggccacaataatggtatttgttggtttaatgaattgtttgttactgttgttgatactac

SEQ ID NO: 132 - DNA, 64 bp, Artificial sequence
  Synthetic oligonucleotide
cacagggccacaataatggaatttgttggcataatcaactgtttgttactgttgttgatactac

SEQ ID NO: 133 - DNA, 64 bp, Artificial sequence
  Synthetic oligonucleotide
cacagggccacaataatggcatatgctggggtaatcaggtatttgttactgttgttgatactac

SEQ ID NO: 134 - DNA, 64 bp, Artificial sequence
  Synthetic oligonucleotide
cacagggccacaataatggcatttgttggggcaaccaattgtttgttactgttgttgatactac

SEQ ID NO: 135 - DNA, 64 bp, Artificial sequence
  Synthetic oligonucleotide
cacagggccacaataatggcatttgttggcataaccagttgtttgttactgttgttgatactac

SEQ ID NO: 136 - DNA, 64 bp, Artificial sequence
  Synthetic oligonucleotide
cacagggccacaataatggcatctgttggtttaatgagctttttgttactgttgttgatactac

SEQ ID NO: 137 - DNA, 64 bp, Artificial sequence
  Synthetic oligonucleotide
cacagggccacaataatggtatttgttggggtaatcaattatttgttactgttgttgatactac

SEQ ID NO: 138 - DNA, 64 bp, Artificial sequence
  Synthetic oligonucleotide
cacagggccacaataatggcatttgctggaataatcagctttttgttactgttgttgatactac

SEQ ID NO: 139 - DNA, 64 bp, Artificial sequence
  Synthetic oligonucleotide
cacagggccacaataatggcatttgttggtttaatgagttatttgttactgttgttgatactac

SEQ ID NO: 140 - DNA, 64 bp, Artificial sequence
  Synthetic oligonucleotide
cacagggccacaataatggtatatgctggtttaatcaattgtttgttactgttgttgatactac

SEQ ID NO: 141 - DNA, 22 bp, Artificial sequence
  Synthetic oligonucleotide
tcgatccctacctacatcttcc

SEQ ID NO: 142 - DNA, 22 bp, Artificial sequence
  Synthetic oligonucleotide
cacacggcagtgaccaacatga

SEQ ID NO: 143 - DNA, 22 bp, Artificial sequence
  Synthetic oligonucleotide
tgtcgcatgctacatacactaa

SEQ ID NO: 144 - DNA, 22 bp, Artificial sequence
  Synthetic oligonucleotide
catacccactctgctacataca

SEQ ID NO: 145 - DNA, 22 bp, Artificial sequence
  Synthetic oligonucleotide
cggcggggccactacatcctct

SEQ ID NO: 146 - DNA, 22 bp, Artificial sequence
  Synthetic oligonucleotide
gcgccatatctacttcccgatc

SEQ ID NO: 147 - DNA, 22 bp, Artificial sequence
  Synthetic oligonucleotide
cctgggcccttctacctgtctc

SEQ ID NO: 148 - DNA, 22 bp, Artificial sequence
  Synthetic oligonucleotide
cagtgcgcactgatccactcca

SEQ ID NO: 149 - DNA, 22 bp, Artificial sequence
  Synthetic oligonucleotide
cagcctgcacaagcgttatcca

SEQ ID NO: 150 - DNA, 22 bp, Artificial sequence
  Synthetic oligonucleotide
tgtttgtgctgtgaatggttcg

SEQ ID NO: 151 - DNA, 22 bp, Artificial sequence
  Synthetic oligonucleotide
tggccacacaagacctgttgaa

SEQ ID NO: 152 - DNA, 22 bp, Artificial sequence
  Synthetic oligonucleotide
cgggggatctacaactgcacca

SEQ ID NO: 153 - DNA, 22 bp, Artificial sequence
  Synthetic oligonucleotide
cgggccgtgtgttccatggtga

SEQ ID NO: 154 - DNA, 22 bp, Artificial sequence
  Synthetic oligonucleotide
ggcacgtggaccaggcgtgtag

SEQ ID NO: 155 - DNA, 22 bp, Artificial sequence
  Synthetic oligonucleotide
catgtccaccgggcacgtgtag

SEQ ID NO: 156 - DNA, 22 bp, Artificial sequence
  Synthetic oligonucleotide
ggcacgggagcggcactaggag

SEQ ID NO: 157 - DNA, 22 bp, Artificial sequence
  Synthetic oligonucleotide
tgtcacaatgtgtcccccacac

SEQ ID NO: 158 - DNA, 22 bp, Artificial sequence
  Synthetic oligonucleotide
cgtgcgcatgcaacattggtga

SEQ ID NO: 159 - DNA, 22 bp, Artificial sequence
  Synthetic oligonucleotide
tgccaagcaaagtgatgtggaa

SEQ ID NO: 160 - DNA, 22 bp, Artificial sequence
  Synthetic oligonucleotide
acacaaggacctctccgtctac

SEQ ID NO: 161 - DNA, 22 bp, Artificial sequence
  Synthetic oligonucleotide
cggtcgatcctgttccaagtgc

SEQ ID NO: 162 - DNA, 22 bp, Artificial sequence
  Synthetic oligonucleotide
tgccactgcttagcggtttccc

SEQ ID NO: 163 - DNA, 22 bp, Artificial sequence
  Synthetic oligonucleotide
gggcacaccttcctctggctcg

SEQ ID NO: 164 - DNA, 23 bp, Artificial sequence
  Synthetic oligonucleotide
gcagctgatgcctctttcccaac

SEQ ID NO: 165 - DNA, 21 bp, Artificial sequence
  Synthetic oligonucleotide
gtggagcagcatcccgcagta

SEQ ID NO: 166 - DNA, 22 bp, Artificial sequence
  Synthetic oligonucleotide
cgtccagatgtgtacaactcag

SEQ ID NO: 167 - DNA, 22 bp, Artificial sequence
  Synthetic oligonucleotide
tgcacgagcatgaacagttaag

SEQ ID NO: 168 - DNA, 22 bp, Artificial sequence
  Synthetic oligonucleotide
ctgggactaaggaagatgttga

SEQ ID NO: 169 - DNA, 22 bp, Artificial sequence
  Synthetic oligonucleotide
ggacggcgccagacgtgtggac

SEQ ID NO: 170 - DNA, 22 bp, Artificial
  Synthetic oligonucleotide
tgcgccaactcgccatacatag

SEQ ID NO: 171 - DNA, 22 bp, Artificial sequence
  Synthetic oligonucleotide
tgggctaggacgacacactccg

SEQ ID NO: 172 - DNA, 22 bp, Artificial sequence
  Synthetic oligonucleotide
tgggcaaacactattttgaggg

SEQ ID NO: 173 - DNA, 22 bp, Artificial sequence
  Synthetic oligonucleotide
tgctgcccgttcccttcgccat

SEQ ID NO: 174 - DNA, 22 bp, Artificial sequence
  Synthetic oligonucleotide
tttggacgaggatcagaggcta

SEQ ID NO: 175 - DNA, 22 bp, Artificial sequence
  Synthetic oligonucleotide
tgtagactgaacagctgtacca

SEQ ID NO: 176 - DNA, 22 bp, Artificial sequence
  Synthetic oligonucleotide
tgcattgccactttgataaacc

SEQ ID NO: 177 - DNA, 22 bp, Artificial sequence
  Synthetic oligonucleotide
cacgccacatcacgaatcccgg

SEQ ID NO: 178 - DNA, 22 bp, Artificial sequence
  Synthetic oligonucleotide
tgcacgtcctctttatcttcgc

SEQ ID NO: 179 - DNA, 22 bp, Artificial sequence
  Synthetic oligonucleotide
ccgatggtgatgccaactctaa

SEQ ID NO: 180 - DNA, 23 bp, Artificial sequence
  Synthetic oligonucleotide
gggacgcaccgccttctcctact

SEQ ID NO: 181 - DNA, 22 bp, Artificial sequence
  Synthetic oligonucleotide
tccgcatggctgtactcaatct

SEQ ID NO: 182 - DNA, 22 bp, Artificial sequence
  Synthetic oligonucleotide
gctgtgtattcatcattgggcc

SEQ ID NO: 183 - DNA, 22 bp, Artificial sequence
  Synthetic oligonucleotide
cgtatcagctcccgaatcataa

SEQ ID NO: 184 - DNA, 23 bp, Artificial sequence
  Synthetic oligonucleotide
ggtaatcaacagtttgaactact

SEQ ID NO: 185 - DNA, 11 bp, Artificial sequence
  Synthetic oligonucleotide
gctggggaaac

SEQ ID NO: 186 - DNA, 14 bp, Artificial sequence
  Synthetic oligonucleotide
aactatttgtactg

SEQ ID NO: 187 - DNA, 13 bp, Artificial sequence
  Synthetic oligonucleotide
gtgttcgctggca

SEQ ID NO: 188 - DNA, 22 bp, Artificial sequence
  Synthetic oligonucleotide
agaaccaaagggcaatcagtta

SEQ ID NO: 189 - DNA, 21 bp, Artificial sequence
  Synthetic oligonucleotide
gtaaccaactatttgtaactg

(19) United States
(12) Patent Application Publication
(10) Pub. No.: US 2010/0167280 A1
(43) Pub. Date: Jul. 1, 2010

(54) OLIGONUCLEOTIDES FOR DISCRIMINATING RELATED NUCLEIC ACID SEQUENCES

(76) Inventors: Ivan Brukner, Montreal (CA); Damian Labuda, Montreal (CA); Maja Krajinovic, Montreal (CA)

Correspondence Address: GOUDREAU GAGE DUBUC, 2000 MCGILL COLLEGE, SUITE 2200, MONTREAL, QC, H3A 3H3, CA

(21) Appl. No.: 12/377,044

(22) PCT Filed: Aug. 10, 2007
(86) PCT No.: PCT/CA2007/001398

§ 371 Date: Oct. 29, 2009

Related U.S. Application Data
(60) Provisional application No. 60/822,153, filed on Aug. 11, 2006.

(51) Int. Cl. C12Q 1/68; C07H 21/04

(52) U.S. Cl. 435/6; 536/23.1

(57) ABSTRACT

An in vitro selection method is described which identifies oligonucleotide probes that discriminate amongst closely related nucleic acid sequences and which involves iterative hybridizations, including subtractive hybridization. Using the method, oligonucleotides are identified which can discriminate among human papilloma virus (HPV) subtypes. Corresponding methods and kits for the detection of nucleic acids are described, which methods and kits may be used in analytical, diagnostic, research and related applications.

### CROSS REFERENCE TO RELATED APPLICATIONS

This application claims the benefit, under 35 U.S.C. §119(e), of U.S. provisional application Ser. No. 60/822,153 filed on Aug. 11, 2006, which is incorporated herein by reference in its entirety.

### FIELD OF THE INVENTION

The invention relates to nucleic acids which may be used for example as probes, methods for their identification and preparation as well as to corresponding methods and kits for their use.

### BACKGROUND OF THE INVENTION

Specific interactions between macromolecules or between macromolecules and their low molecular weight ligands play an important role in all biological processes. Specific interactions also find practical applications to elaborate research tools in molecular biology, medicine and in molecular diagnostics. The specificity, describing the ability to discriminate between different ligands, is often equated with the affinity between the interacting molecules (Lomakin and Frank-Kamenetskii. 1998. Journal of Molecular Biology, 276(1): 57-70). A ligand of sufficiently high-affinity is expected to be highly specific for its target and the high affinity/high specificity paradigm was considered applicable to virtually all interacting systems. However, this paradigm does not as easily apply to nucleic acids: nucleic acid hybridization, fundamental to many techniques in molecular genetics. Although it is true that the interaction between nucleic acid strands becomes stronger with each additional base-pair and that a longer probe sequence most precisely defines target nucleic acid than a shorter sequence, in practice, the ability of an oligo- or a polynucleotide to discriminate among closely related sequences through hybridization actually decreases as a function of sequence length. Cross-hybridization of similar but non-identical sequences becomes more probable with longer sequences. In Polymerase Chain Reaction (PCR), for example, to avoid false priming, the annealing of primers is usually carried out at the highest possible temperature that maximizes the stability gap between complementary and mismatched duplexes. However, if sequences that are to be distinguished are similar, the difference in their binding energies is small restricting the window of adjustable experimental conditions that would allow discrimination between all potentially reacting species. Finding such conditions becomes problematic in multiplex applications, when many probes and/or many targets are considered simultaneously (Simard et al., 1991. Nucleic Acids Res., 9: 2501; Gharizadeh et al., 2003, Nucleic Acids Res 31: e146).

There thus remains a need for improved nucleic acid probes for example having an enhanced power of detection of small differences between target sequence motifs.

The present description refers to a number of documents, the content of which is herein incorporated by reference in their entirety.

### SUMMARY OF THE INVENTION

The invention relates to oligonucleotides (e.g., nucleic acid probes), to methods of generating said oligonucleotides, to uses of said oligonucleotides and to corresponding kits and collections of oligonucleotides. The oligonucleotides, methods, uses, kits and collections of the invention are particularly useful (e.g., as probes) for discriminating between closely related or similar nucleic acids.

Accordingly, in an aspect, the invention provides a method for identifying or preparing an oligonucleotide for discriminating a first nucleic acid from a second nucleic acid, said method comprising:
(a) hybridizing said first nucleic acid with a pool of oligonucleotides in a hybridization mixture, said oligonucleotides comprising a random nucleotide sequence flanked by primer recognition sequences;
(b) removing oligonucleotides which are not bound to said first nucleic acid from said hybridization mixture;
(c) dissociating bound oligonucleotides from said first nucleic acid;
(d) amplifying said bound oligonucleotides using primers capable of binding to said primer recognition sequences to obtain amplified oligonucleotide duplexes comprising a first strand corresponding to said bound oligonucleotides and a second strand corresponding to the complement of said bound oligonucleotides;
(e) treating said duplexes to remove or degrade said second strand to obtain single-stranded amplified oligonucleotides;
(f) repeating (a) to (e), wherein said pool of oligonucleotides of (a) is the amplified oligonucleotides obtained in (e) in each cycle thereby to obtain further amplified oligonucleotides; and
(g) repeating (a) to (e), wherein said hybridization is performed in the presence of said second nucleic acid;
   wherein an oligonucleotide comprising the random nucleotide sequence of said further amplified oligonucleotides is capable of discriminating said first nucleic acid from said second nucleic acid.

In embodiments, said repeating step (f) is performed at least 1, 2, 3 or 4 times, in a further embodiment, 4 times.

In embodiments, said repeating step (g) is performed at least 1, 2, or 3 times, in a further embodiment, 3 times.

In an embodiment, the above-mentioned method further comprises selecting an oligonucleotide from said further amplified oligonucleotides on the basis of its preferential binding to said first nucleic acid relative to said second nucleic acid.

In an embodiment, said hybridization is performed in the presence of a blocking agent capable of inhibiting binding of said primer recognition sequences to said first nucleic acid. In a further embodiment, said blocking agent is an oligonucleotide capable of binding said primer recognition sequences.

In an embodiment, said first nucleic acid is derived from a pathogen. In a further embodiment, said pathogen is selected from a eukaryote, prokaryote and a virus. In a further embodiment, said virus is human papillomavirus (HPV).

In an embodiment, said first and second nucleic acids are derived from different subtypes of HPV. In a further embodiment, said subtypes are selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

In an embodiment, said first nucleic acid or said oligonucleotide is bound to a solid support.

In an embodiment, the random nucleotide sequence of said further amplified oligonucleotides is not exactly complementary to said first nucleic acid. In a further embodiment, the random nucleotide sequence of said further amplified oligonucleotides comprises at least 1 mismatch relative to said first nucleic acid.

In an embodiment, said amplification is performed using polymerase chain reaction (PCR) or isothermal amplification.

In an embodiment, said dissociation is performed by incubation at an elevated temperature relative to said hybridization. In an embodiment, the above-mentioned temperature is a temperature above the melting temperature (Tm). In a further embodiment, said elevated temperature is at least about 85° C.

In an embodiment, said treatment is with an exonuclease capable of selective degradation of said second strand. In a further embodiment, said selectivity is based on 5′-terminal phosphorylation of said strand. In a further embodiment, said exonuclease is lambda (λ) exonuclease.

In another aspect, the invention provides an oligonucleotide capable of discriminating a first nucleic acid from a second nucleic acid, wherein said oligonucleotide is not exactly complementary to said first nucleic acid. In an embodiment, said oligonucleotide comprises at least 1 mismatch relative to said first nucleic acid. In a further embodiment, said first nucleic acid is derived from a pathogen. In a further embodiment, said pathogen is selected from a eukaryote, prokaryote and a virus. In a further embodiment, said virus is human papillomavirus (HPV).

In an embodiment, said first and second nucleic acids are derived from different subtypes of HPV. In a further embodiment, said subtypes are selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

In an embodiment, the above-mentioned oligonucleotide comprises a nucleotide sequence selected from SEQ ID NOs: 1-43, 100-104 and 116. In a further embodiment, said oligonucleotide comprises a sequence and is capable of selectively detecting an HPV subtype as set forth in FIG. 11.

In another aspect, the invention provides an oligonucleotide comprising a nucleotide sequence selected from SEQ ID NOs: 1-43, 100-104 and 116.

In yet another aspect, the present invention provides a collection of two or more oligonucleotides comprising a nucleotide sequence selected from SEQ ID NOs: 1-43, 100-104 and 116. In an embodiment, the above-mentioned oligonucleotides are immobilized on a substrate (e.g., at discrete locations on the substrate). In another embodiment, the above-mentioned oligonucleotides are conjugated to a detectable marker. In a further embodiment, the above-mentioned detectable marker is a fluorescent moiety. In another embodiment, the above-mentioned oligonucleotides are hybridizable array elements in a microarray.

In another aspect, the present invention provides an array comprising the above-mentioned oligonucleotide or the above-mentioned collection of two or more oligonucleotides.

In another aspect, the invention provides a method for detecting the presence of a first nucleic acid in a sample, said method comprising contacting the above-mentioned oligonucleotide with said sample under conditions permitting selective hybridization of said oligonucleotide to said first nucleic acid, wherein selective hybridization is indicative that said first nucleic acid is present in said sample. In an embodiment, said first nucleic acid is derived from a pathogen and said method is for detection of said pathogen in a sample. In a further embodiment, said sample is a biological sample derived from a subject and said method is for detection of said pathogen in said subject. In a further embodiment, said method is for diagnosing a disease or condition associated with said pathogen in said subject. In a further embodiment, said pathogen is selected from a eukaryote, prokaryote and a virus. In a further embodiment, said virus is human papillomavirus (HPV). In a further embodiment, said subject is a mammal. In a further embodiment, said mammal is a human.

In an embodiment, the above-mentioned method is for detecting the presence of a subtype of HPV. In a further embodiment, said subtype is selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

In an embodiment, the above-mentioned oligonucleotide is bound to a solid support. In another embodiment, the above-mentioned first nucleic acid is labelled with a detectable marker. In a further embodiment, the above-mentioned detectable marker is a fluorescent moiety.

In another aspect, the invention provides a kit for detecting the presence of a first nucleic acid in a sample, said kit comprising an oligonucleotide as described herein.

In an embodiment, said kit comprises:

(a) an oligonucleotide as described herein; and

(b) means for detecting selective hybridization of said oligonucleotide to said first nucleic acid.

In an embodiment, the above-mentioned kit further comprises instructions setting forth the above-mentioned method.

In a further embodiment, said first nucleic acid is derived from a pathogen and said kit is for detecting the presence of said pathogen in said sample. In a further embodiment, said sample is a biological sample derived from a subject and said kit is for detection of said pathogen in said subject.

In an embodiment, the above-mentioned kit is for diagnosing a disease or condition associated with said pathogen in said subject. In a further embodiment, said pathogen is selected from a eukaryote, prokaryote and a virus. In a further embodiment, said virus is human papillomavirus (HPV).

In an embodiment, the above-mentioned kit is for detecting the presence of a subtype of HPV. In a further embodiment, said subtype is selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

In an embodiment, the above-mentioned kit comprises the above-mentioned oligonucleotide.

The invention further provides an oligonucleotide identified or prepared by the above-mentioned method.

In embodiments, the above-mentioned oligonucleotide is selected from:

an oligonucleotide comprising SEQ ID NO: 1, 2, 100 or 116 and which is capable of selectively detecting HPV 6;
an oligonucleotide comprising SEQ ID NO: 3, 4 or 101 and which is capable of selectively detecting HPV 11;
an oligonucleotide comprising SEQ ID NO: 5 and which is capable of selectively detecting HPV 13;
an oligonucleotide comprising SEQ ID NO: 6 or 102 and which is capable of selectively detecting HPV 16;
an oligonucleotide comprising SEQ ID NO: 7 or 103 and which is capable of selectively detecting HPV 18;
an oligonucleotide comprising SEQ ID NO: 8 and which is capable of selectively detecting HPV 26;
an oligonucleotide comprising SEQ ID NO: 9 and which is capable of selectively detecting HPV 30;
an oligonucleotide comprising SEQ ID NO: 10 and which is capable of selectively detecting HPV 31;
an oligonucleotide comprising SEQ ID NO: 11 and which is capable of selectively detecting HPV 33;
an oligonucleotide comprising SEQ ID NO: 12 and which is capable of selectively detecting HPV 34;
an oligonucleotide comprising SEQ ID NO: 13 and which is capable of selectively detecting HPV 39;
an oligonucleotide comprising SEQ ID NO: 14, 15 or 16 and which is capable of selectively detecting HPV 39;
an oligonucleotide comprising SEQ ID NO: 17 and which is capable of selectively detecting HPV 40;
an oligonucleotide comprising SEQ ID NO: 18 and which is capable of selectively detecting HPV 42;
an oligonucleotide comprising SEQ ID NO: 19 and which is capable of selectively detecting HPV 43;
an oligonucleotide comprising SEQ ID NO: 20 and which is capable of selectively detecting HPV 44;
an oligonucleotide comprising SEQ ID NO: 21 and which is capable of selectively detecting HPV 45;
an oligonucleotide comprising SEQ ID NO: 22 and which is capable of selectively detecting HPV 51;
an oligonucleotide comprising SEQ ID NO: 23 and which is capable of selectively detecting HPV 52;
an oligonucleotide comprising SEQ ID NO: 24 and which is capable of selectively detecting HPV 53;
an oligonucleotide comprising SEQ ID NO: 25 and which is capable of selectively detecting HPV 54;
an oligonucleotide comprising SEQ ID NO: 26 and which is capable of selectively detecting HPV 55;
an oligonucleotide comprising SEQ ID NO: 27 and which is capable of selectively detecting HPV 56;
an oligonucleotide comprising SEQ ID NO: 28 and which is capable of selectively detecting HPV 58;
an oligonucleotide comprising SEQ ID NO: 29 and which is capable of selectively detecting HPV 59;
an oligonucleotide comprising SEQ ID NO: 30 and which is capable of selectively detecting HPV 61;
an oligonucleotide comprising SEQ ID NO: 31 and which is capable of selectively detecting HPV 62;
an oligonucleotide comprising SEQ ID NO: 32 and which is capable of selectively detecting HPV 64;
an oligonucleotide comprising SEQ ID NO: 33 and which is capable of selectively detecting HPV 66;
an oligonucleotide comprising SEQ ID NO: 34 and which is capable of selectively detecting HPV 67;
an oligonucleotide comprising SEQ ID NO: 35 and which is capable of selectively detecting HPV 68;
an oligonucleotide comprising SEQ ID NO: 36 and which is capable of selectively detecting HPV 69;
an oligonucleotide comprising SEQ ID NO: 37 and which is capable of selectively detecting HPV 70;
an oligonucleotide comprising SEQ ID NO: 38 and which is capable of selectively detecting HPV 72;
an oligonucleotide comprising SEQ ID NO: 39 and which is capable of selectively detecting HPV 73;
an oligonucleotide comprising SEQ ID NO: 40 and which is capable of selectively detecting HPV 74;
an oligonucleotide comprising SEQ ID NO: 41 and which is capable of selectively detecting HPV MM4;
an oligonucleotide comprising SEQ ID NO: 42 and which is capable of selectively detecting HPV MM7;
an oligonucleotide comprising SEQ ID NO: 43 and which is capable of selectively detecting HPV MM8; and
an oligonucleotide comprising SEQ ID NO: 104 and which is capable of selectively detecting HPV 31 and/or 33.

In embodiments, the above-mentioned oligonucleotide is selected from:

an oligonucleotide comprising SEQ ID NO: 1, 2, 100 or 116 and wherein the first nucleic acid is derived from HPV 6;
an oligonucleotide comprising SEQ ID NO: 3, 4 or 101 and wherein the first nucleic acid is derived from HPV 11;
an oligonucleotide comprising SEQ ID NO: 5 and wherein the first nucleic acid is derived from HPV 13;
an oligonucleotide comprising SEQ ID NO: 6 or 102 and wherein the first nucleic acid is derived from HPV 16;
an oligonucleotide comprising SEQ ID NO: 7 or 103 and wherein the first nucleic acid is derived from HPV 18;
an oligonucleotide comprising SEQ ID NO: 8 and wherein the first nucleic acid is derived from HPV 26;
an oligonucleotide comprising SEQ ID NO: 9 and wherein the first nucleic acid is derived from HPV 30;
an oligonucleotide comprising SEQ ID NO: 10 and wherein the first nucleic acid is derived from HPV 31;
an oligonucleotide comprising SEQ ID NO: 11 and wherein the first nucleic acid is derived from HPV 33;
an oligonucleotide comprising SEQ ID NO: 12 and wherein the first nucleic acid is derived from HPV 34;
an oligonucleotide comprising SEQ ID NO: 13 and wherein the first nucleic acid is derived from HPV 39;
an oligonucleotide comprising SEQ ID NO: 14, 15 or 16 and wherein the first nucleic acid is derived from HPV 39;
an oligonucleotide comprising SEQ ID NO: 17 and wherein the first nucleic acid is derived from HPV 40;
an oligonucleotide comprising SEQ ID NO: 18 and wherein the first nucleic acid is derived from HPV 42;
an oligonucleotide comprising SEQ ID NO: 19 and wherein the first nucleic acid is derived from HPV 43;
an oligonucleotide comprising SEQ ID NO: 20 and wherein the first nucleic acid is derived from HPV 44;
an oligonucleotide comprising SEQ ID NO: 21 and wherein the first nucleic acid is derived from HPV 45;
an oligonucleotide comprising SEQ ID NO: 22 and wherein the first nucleic acid is derived from HPV 51;
an oligonucleotide comprising SEQ ID NO: 23 and wherein the first nucleic acid is derived from HPV 52;
an oligonucleotide comprising SEQ ID NO: 24 and wherein the first nucleic acid is derived from HPV 53;
an oligonucleotide comprising SEQ ID NO: 25 and wherein the first nucleic acid is derived from HPV 54;
an oligonucleotide comprising SEQ ID NO: 26 and wherein the first nucleic acid is derived from HPV 55;
an oligonucleotide comprising SEQ ID NO: 27 and wherein the first nucleic acid is derived from HPV 56;
an oligonucleotide comprising SEQ ID NO: 28 and wherein the first nucleic acid is derived from HPV 58;
an oligonucleotide comprising SEQ ID NO: 29 and wherein the first nucleic acid is derived from HPV 59;
an oligonucleotide comprising SEQ ID NO: 30 and wherein the first nucleic acid is derived from HPV 61;
an oligonucleotide comprising SEQ ID NO: 31 and wherein the first nucleic acid is derived from HPV 62;
an oligonucleotide comprising SEQ ID NO: 32 and wherein the first nucleic acid is derived from HPV 64;
an oligonucleotide comprising SEQ ID NO: 33 and wherein the first nucleic acid is derived from HPV 66;
an oligonucleotide comprising SEQ ID NO: 34 and wherein the first nucleic acid is derived from HPV 67;
an oligonucleotide comprising SEQ ID NO: 35 and wherein the first nucleic acid is derived from HPV 68;
an oligonucleotide comprising SEQ ID NO: 36 and wherein the first nucleic acid is derived from HPV 69;
an oligonucleotide comprising SEQ ID NO: 37 and wherein the first nucleic acid is derived from HPV 70;
an oligonucleotide comprising SEQ ID NO: 38 and wherein the first nucleic acid is derived from HPV 72;
an oligonucleotide comprising SEQ ID NO: 39 and wherein the first nucleic acid is derived from HPV 73;
an oligonucleotide comprising SEQ ID NO: 40 and wherein the first nucleic acid is derived from HPV 74;
an oligonucleotide comprising SEQ ID NO: 41 and wherein the first nucleic acid is derived from HPV MM4;
an oligonucleotide comprising SEQ ID NO: 42 and wherein the first nucleic acid is derived from HPV MM7;
an oligonucleotide comprising SEQ ID NO: 43 and wherein the first nucleic acid is derived from HPV MM8; and
an oligonucleotide comprising SEQ ID NO: 104 and wherein the first nucleic acid is derived from HPV 31 and/or 33.

In embodiments, the above-mentioned methods of detection or diagnosis are in vitro methods of detection or diagnosis.

The present invention further provides a kit for identifying an oligonucleotide (e.g., which can be used as a nucleic acid probe) for discriminating a first nucleic acid from a second nucleic acid in accordance with the above-mentioned method. In an embodiment, the kit comprises the above-mentioned pool of oligonucleotides. In a further embodiment, the kit comprises instructions setting forth the above-mentioned method.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows target short PCR fragment, SPF, of distinct HPV subtypes. Twenty-two nucleotide long amplified sequence is flanked by sequences used to anchor the PCR primers as indicated (A) and the matrix of pairwise nucleotide differences between the considered SPF sequences (B). Bt and 6-FAM denote 5′ terminal modifications with biotin and 6-carboxyfluorescein, respectively. Dots indicate identity with the upper sequence;

FIG. 2 shows binding of probes to their cognate and non-cognate targets. In A) binding of the pools of probes, PPs, obtained after five rounds of iterative hybridization (5+); in B) of PPs after they were submitted to three additional rounds of subtractive hybridizations (5+3−); and in C) of the full 22-nucleotide long complements of the targets. All probes were labelled with 6-FAM at their 5′ terminus to allow quantification of the extent of hybridization, expressed in arbitrary units and corresponding to the bound measured fluorescence signal (RFU=relative fluorescence units);

FIG. 3 shows competitive titration of the immobilized HPV-16 target (T16). T16 was hybridized: in A) with its 6-FAM-labelled complement; in B) with its PP16 (5+3−), and in C) with its cloned probe CP16 (see FIG. 4 for the corresponding sequence). The bound fluorescence was chased by increasing concentrations of the non-biotinylated cognate (T16) or each of the non-biotinylated non-cognate target oligonucleotides. The effective concentration EC₅₀ of the competitive target oligonucleotides required to reduce binding by 50% was calculated, expressed as log EC₅₀. Δ log EC₅₀ is a difference between the log EC₅₀ values obtained for T16 and a competitive non-cognate target as indicated;

FIG. 4 shows cloned probes (CPs) in the context of their cognate target sequences. A) Differences in the SPF targets are highlighted whereas dots indicate matches between targets and the reverse complement of the corresponding CPs. Note that the CP sequences are flanked by priming sequences, notably those shown as the constant sequence fragments in the structure of ROM22 in Example 1, below, which are not shown in this figure. B) Nucleotide sequence of the probes and their corresponding SEQ ID NOs;

FIG. 5 shows binding of the individual cloned probes: in A) to the immobilized cognate and non-cognate HPV targets, and in B) the same binding, but in reverse format instead, i.e. of the free PCR amplified tested HPV targets to the cognate and non-cognate immobilized cloned probes from FIG. 4;

FIG. 6 shows modified forward and reverse universal primers amplifying GP5+/6+ region of HPV (reference: between 6647 and 6740, GI: 333031, GenBank Accession No. K02718). Modification was introduced to equilibrate the priming capacity among different types and tested on L1 HPV-containing plasmids, having slightly different primer-binding sequences (HPV 6, 11, 16, 18, 31, 33 and 52) and corresponding clinical samples. The forward primer GP5M was design to contain degenerative nucleotides at all variable positions along GP5+ primer-binding site, while GP6M was binding to GP6+ binding site and synthesized in four variants (GP6.1-4) where each variant have relevant combination of nucleotides at first 5 positions of 3′ end of the reverse primer;

FIG. 7 shows alignments of 39 HPV target sequences between positions 6647 and 6740 as in HPV16 (GI: 333031, GenBank Accession No. K02718), as obtained by ClustalW (Chema et al., (2003) Nucleic Acids Res 31 (13):3497-500; available at http://www.ebi.ac.uk/clustalw/);

FIG. 8 shows hybridization of the selected pooled probes, PPs (A) and of the individual cloned probes, CPs (B) with each of the HPV type. PPs were obtained after five rounds of positive and 2 rounds of subtractive hybridization (5+2−). CPs were selected based on the best performing 2 to 10 clones during CP validation, using a signal-noise hybridization threshold≧3. Gray scale represents relative extent of hybridization intensities;

FIG. 9 shows sequences of the reverse complement of selected cloned probes, CP, in the context of their cognate target sequences (GP5+/6+ amplicon). The probe-binding site to each target is highlighted in grey, while the full probe reverse complement sequence is written below the target-binding site. The full-matches are underlined. Note that the CP sequences are flanked by priming sequences that are not shown here;

FIG. 10 shows partial sequence alignment of CP33 with its specific and nine similar HPV targets. The mismatch that breaks an elongated stretch of complementarity between CP33 and its target is highlighted in grey. Dots represent nucleotide identity with the uppermost CP33 and different sequences below. Note that targets are in the usual 5′-3′ orientation, while upper CP33 is represented by its antisense strand (reverse complement) to facilitate the comparison;

FIG. 11 shows correspondence of SEQ ID NOs: of HPV subtype-specific nucleic acid probe sequences described herein;

FIG. 12 shows A) alignments of the reverse complement of Cloned Probe SPF HPV16 (CP_{—}16_SPF_{—}50_Celsius (rc)) which is able to discriminate SPF amplicon of HPV16 from all other SPF amplicons illustrated in FIG. 12. Dots represent full match complementarities between the HPV target sequences and the reverse complement sequence of Cloned Probe SPF HPV16. The HPV subtype is indicated on the left side. Selection of probe (originated from random segment) was performed as described in Example 1, except that the temperature of hybridization and washing was kept at 50° C. The target was SPF fragment of HPV16, while the non-intended targets are the group of 23 other HPV subtypes illustrated in FIG. 12. B) Nucleotide sequence of cloned probe SPF HPV16 (CP_{—}16_SPF_{—}50_Celsius) and its corresponding SEQ ID NO;

FIG. 13 shows performance of 39 CPs with HPV16 target. Probes are in the same linear order as HPV targets illustrated in FIG. 7;

FIG. 14 shows HPV typing of pre-characterized clinical samples containing HPV6 and HPV16 to the array of 39 immobilized type-specific CPs. (A) the arrangement of CPs; (B) hybridization with HPV6; (C) hybridization with HPV16. Arrows indicate the orientation of the probes array; and

FIG. 15 shows a schematic presentation of iterative hybridizations, composed of two steps: forward or positive (left panel) and subtractive hybridizations (right panel). Note that intended targets are attached to the solid support, while non-intended targets are free in solution.

### DETAILED DESCRIPTION OF THE INVENTION

The invention relates to oligonucleotides (e.g., nucleic acid probes), methods for their identification and preparation, and corresponding uses, methods, kits, collections and related products.

To aid in understanding the invention and its preferred embodiments, various definitions are provided. Other scientific and technical terms used herein have the same meaning as commonly understood by those skilled in the relevant art. General definitions of terms may be found in, e.g., Dictionary of Microbiology and Molecular Biology, 2^{nd} ed. (Singleton et al., 1994, John Wiley & Sons, New York, N.Y.) or The Harper Collins Dictionary of Biology (Hale & Marham, 1991, Harper Perennial, New York, N.Y.). Unless otherwise described, the techniques employed or contemplated herein are standard methodologies that are well known to one of ordinary skill in the art.

Accordingly, in a first aspect, the present invention provides a method for identifying or preparing an oligonucleotide (e.g., which can be used as a probe) for discriminating a first nucleic acid from a second nucleic acid, said method comprising:
(a) hybridizing said first nucleic acid with a pool of oligonucleotides in a hybridization mixture, said oligonucleotides comprising a random nucleotide sequence flanked by primer recognition sequences;
(b) removing oligonucleotides which are not bound to said first nucleic acid from said hybridization mixture;
(c) dissociating bound oligonucleotides from said first nucleic acid;
(d) amplifying said bound oligonucleotides using primers capable of binding to said primer recognition sequences to obtain amplified oligonucleotide duplexes comprising a first strand corresponding to said bound oligonucleotides and a second strand corresponding to the complement of said bound oligonucleotides;
(e) treating said duplexes to remove or degrade said second strand to obtain single-stranded amplified oligonucleotides;
(f) repeating (a) to (e), wherein said pool of oligonucleotides of (a) is the amplified oligonucleotides obtained in (e) in each cycle thereby to obtain further amplified oligonucleotides; and
(g) repeating (a) to (e), wherein said hybridization is performed in the presence of said second nucleic acid;
   wherein the random nucleotide sequence of said further amplified oligonucleotides is capable of discriminating said first nucleic acid from said second nucleic acid.

In an embodiment, said repeating step (f) is performed at least 1 time, in a further embodiment, at least 2 times, in yet a further embodiment, at least 3 times, in yet a further embodiment, at least 4 times.

In an embodiment, said repeating step (g) is performed at least 1 time, in a further embodiment, at least 2 times, in a further embodiment, at least 3 times. Such repeating step (g) provides a subtractive hybridization.

In further embodiments, the concentration or amount of said second nucleic acid may be increased from a cycle of repeating step (g) to a subsequent or later cycle of repeating step (g).

The random nucleotide sequences identified via the method may for example be separated into individual clones, for example via introduction of the random nucleotide sequences into a suitable vector (e.g., a plasmid vector) and the selection of individual clones.

A typical application of the method described herein is for identifying or preparing an oligonucleotide for discriminating a desired or intended target nucleic acid (e.g., the first nucleic acid noted herein) from other, undesired or non-intended non-target nucleic acids (e.g., the second nucleic acid noted herein). One of the advantages of the above-mentioned method is the capacity of identifying or preparing an oligonucleotide for discriminating nucleic acids which share sequence similarities, for example similar nucleic acid sequences from different organisms (e.g. orthologous genes), variants (e.g. polymorphisms, different alleles) of a given nucleic acid sequence, nucleic acid sequences derived from genes belonging to the same family or nucleic acids derived from subtypes of a given organism (e.g. virus, bacteria, parasites). In an embodiment, the first and second nucleic acids do not differ by more than 10 bases per 20 bases; in a further embodiment, do not differ by more than 9 bases per 20 bases; in a further embodiment, do not differ by more than 8 bases per 20 bases; in a further embodiment, do not differ by more than 7 bases per 20 bases; in a further embodiment, do not differ by more than 6 bases per 20 bases; in a further embodiment, do not differ by more than 5 bases per 20 bases; in a further embodiment, do not differ by more than 4 bases per 20 bases; in a further embodiment, do not differ by more than 3 bases per 20 bases; in a further embodiment, do not differ by more than 2 bases per 20 bases; in a further embodiment, do not differ by more than 1 bases per 20 bases. In further embodiments, the first and second nucleic acids do not differ by more than 10, 9, 8, 7, 6, 5, 4, 3, 2 or 1 base(s).

As used herein, “nucleic acid” refers to a multimeric compound (oligomer or polymer) comprising nucleosides or nucleoside analogs which have nitrogenous bases, or base analogs, and which are linked together by phosphodiester bonds or other known linkages to form a polynucleotide. Nucleic acids include conventional ribonucleic acid (RNA), deoxyribonucleic acid (DNA), or chimeric DNA-RNA, and analogs thereof. A nucleic acid “backbone” may be made up of a variety of linkages, including one or more of sugar-phosphodiester linkages, peptide-nucleic acid bonds (in “peptide nucleic acids” or PNAs, see PCT No. WO 95/32305), phosphorothioate linkages, methylphosphonate linkages, or combinations thereof. Sugar moieties of the nucleic acid may be either ribose or deoxyribose, or similar compounds having known substitutions, e.g., 2′ methoxy substitutions and 2′ halide substitutions (e.g., 2′-F). Nitrogenous bases may be conventional bases (A, G, C, T, U), analogs thereof (e.g., inosine; Adams et al., The Biochemistry of the Nucleic Acids, pp. 5-36, 11^{th} ed., 1992), derivatives of purine or pyrimidine bases (e.g., N⁴-methyl deoxygaunosine, deaza- or aza-purines, deaza- or aza-pyrimidines, pyrimidine bases having substituent groups at the 5 or 6 position, purine bases having an altered or replacement substituent at the 2, 6 and/or 8 position, such as 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines, and pyrazolo-compounds, such as unsubstituted or 3-substituted pyrazolo[3,4-d]pyrimidine; U.S. Pat. Nos. 5,378,825, 6,949,367 and PCT No. WO 93/13121). Nucleic acids may include “abasic” residues in which the backbone does not include a nitrogenous base for one or more residues (U.S. Pat. No. 5,585,481). A nucleic acid may comprise only conventional sugars, bases, and linkages as found in RNA and DNA, or may include conventional components and substitutions (e.g., conventional bases linked by a 2′ methoxy backbone, or a nucleic acid including a mixture of conventional bases and one or more base analogs). Nucleic acids also include “locked nucleic acids” (LNA), an analogue containing one or more LNA nucleotide monomers with a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA) (Vester et al., 2004, Biochemistry 43(42):13233-41). Synthetic methods for making nucleic acids in vitro are well known in the art.

The term “oligonucleotide” (e.g. primer, probe) refers to a nucleic acid molecule of any length, but having generally less than 1,000 residues, including those in a size range having a lower limit of about 2 to 5 nucleotides. Preferred oligonucleotides fall in a size range having a lower limit of about 5 to about 15 nucleotides and an upper limit of about 60 to about 150 nucleotides. In an embodiment, oligonucleotides are in a size range of about 15 to 100 nucleotides. In a further embodiment, oligonucleotides are in a size range of about 15 to about 50 nucleotides. In a further embodiment, oligonucleotides are in a size range of about 20 to about 30 nucleotides. The oligonucleotides may be purified from naturally occurring sources, or preferably prepared by established oligonucleotide synthesis methods known in the art. Examples of such methods include synthetic methods such as the cyanoethyl phosphoramidite, phosphotriester, and phosphite-triester methods (Narang et al., 1980. Meth. Enzymol. 65:610-620; Ikuta et al., 1984. Ann. Rev. Biochem. 53:323-356) or the preparation of protein nucleic acid molecules (Nielsen et al., 1994. Bioconj. Chem. 5:3-7). Other methods include typical enzymatic digestion followed by nucleic acid fragment isolation. In an embodiment, the oligonucleotides are prepared by the method described herein.

The oligonucleotide (primer and/or probe) of the present invention may be modified, for example by the inclusion of a fluorescent molecule, such as 6-carboxyflorescein (6-FAM). Other modifications may be utilized, such as those which confer greater stability and nuclease resistance to the oligonucleotide. A preferred modification of this type is the inclusion of phosphorothioate linkages, for example, the first two bonds from the 3′ end of degenerative/random primers can contain phosphorothioate linkages.

A “nucleic acid probe” or “probe” refers to an oligonucleotide that interacts specifically with a target sequence in a nucleic acid, such as an amplified sequence, under conditions that promote such interaction, to allow detection of the target sequence or amplified nucleic acid. Detection may either be direct (i.e., resulting from a probe hybridizing directly to the target or amplified nucleic acid) or indirect (i.e., resulting from a probe hybridizing to an intermediate molecular structure that links the probe to the target or amplified nucleic acid). Such interactions include classical hybridization of complementary sequences, as well as non-Watson-Crick types of interactions. A probe's “target” generally refers to a sequence within (i.e., a subset of) a (e.g., an amplified) nucleic acid sequence which hybridizes specifically to at least a portion of a probe. In an embodiment, a probe is a nucleic acid having generally less than about 1,000 residues, including those in a size range having a lower limit of about 2 to about 5 nucleotides. In an embodiment, the probes fall in a size range having a lower limit of about 5 to about 15 nucleotides and an upper limit of about 60 to about 150 nucleotides. In a further embodiment, probes are in a size range of about 10 to about 100 nucleotides. In a further embodiment, probes are in a size range of about 15 to about 50 nucleotides. In a further embodiment, probes are in a size range of about 20 to about 30 nucleotides.

In an embodiment, the oligonucleotide and/or nucleic acid of the present invention can be labelled. A “label” refers to a molecular moiety or compound that can be detected or can lead to a detectable response. A label can be joined directly or indirectly to a nucleic acid probe. Direct labeling can occur through bonds or interactions that link the label to the probe, including covalent bonds or non-covalent interactions, e.g., hydrogen bonding, hydrophobic and ionic interactions, or formation of chelates or coordination complexes. Indirect labeling can occur through use of a bridging moiety or “linker” which is/are either directly or indirectly labelled, and which may amplify a detectable signal. Labels can be any known detectable moiety, e.g. radionuclides, ligands, enzyme or enzyme substrate, reactive group, or chromophore, such as a dye, bead, or particle that imparts a detectable color, luminescent compounds (e.g., bioluminescent, phosphorescent or chemiluminescent labels) and fluorescent compounds. In an embodiment, the label on a labelled probe is detectable in a homogeneous assay system, i.e., bound labelled probe in a mixture containing unbound probe exhibits a detectable change, such as stability or differential degradation, compared to unbound probe. Synthesis and methods of attaching labels to nucleic acids and detecting labels are well known (see Sambrook et al., Molecular Cloning, A Laboratory Manual, 2^{nd} ed. (Cold Spring Harbor Laboratory Press, Cold Spring Harbor, N.Y., 1989), Chapter 10; U.S. Pat. No. 5,658,737; U.S. Pat. No. 5,656,207; U.S. Pat. No. 5,547,842; U.S. Pat. No. 5,283,174; U.S. Pat. No. 4,581,333; and European Pat. App. Pub. No. 0 747 706).

In an embodiment, particularly for use in the methods of the invention, the oligonucleotides of the present invention comprise “primer recognition sequences” (or “flanking primer-anchoring segments”) and a random sequence segment. The random (sometimes also referred to as degenerate or degenerative) sequence segment is not specifically designed to be complementary to a particular template sequence, and is for example designed based on various permutations and combinations of the common nucleotide bases (e.g., A, C, G, T/U) at any given position therein. In a preferred embodiment, the primer recognition sequences and the random sequence segment are in the following configuration:
primer recognition sequence →random sequence segment →primer recognition sequence

Any suitable nucleic acid sequence may be used as a primer recognition sequence, and is generally a nucleic acid sequence which is not normally contiguous with the target nucleic acid sequence but could be from the same source (e.g., same organism) or from a heterologous source (e.g., different organism or synthetic/recombinant sources) such as DNA from a natural source (e.g., a fragment of DNA isolated from a cell) to other, e.g., synthetic, sources, such as poly(dA-dT), polydAT, poly dG-dC, poly dGC or similar polymers. In embodiments, the flanking primer-anchoring segments may range in size from about 15 to about 40 bases or more in length.

The random sequence segment may range in size from about 5 to about 100 bases or more in length. In an embodiment, the random sequence segment ranges in size from about 10 to about 100 nucleotides. In a further embodiment, the random sequence segment ranges in size from about 15 to about 50 nucleotides. In a further embodiment, the random sequence segment ranges in size from about 20 to about 30 nucleotides.

In an aspect, a nucleic acid of the invention is “isolated” or “substantially purified”. An “isolated” nucleic acid as used herein is defined as a nucleic acid that is separated from the environment in which it naturally occurs and/or that is free of the majority of the nucleic acids that are present in the environment in which it naturally occurs, for example including a nucleotide sequence which is contiguous with a nucleic acid sequence with which it is not contiguous in nature. For example, an isolated nucleic acid is substantially free from contaminants. Those skilled in the art would readily understand that the nucleic acid of the invention may be chemically synthesized or generated from a natural source. A nucleic acid of the invention may also be “synthetic”, which refers to its preparation by synthesis rather than e.g., isolation from a natural source.

In a further embodiment, nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny or cell or genome with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention.

As used herein, the term “amplification” refers to an in vitro method for obtaining multiple copies of a target sequence, its complement, or fragments of a target sequence, as well as for increasing the number of copies of an oligonucleotide of the invention. Amplification of “fragments” refers to production of an amplified nucleic acid that contains less than the complete target region sequence or its complement. For example, a complete gene may be referred to as a target sequence for an assay, but amplification may make copies of a smaller sequence (e.g., about 40 to about 3000 nucleotides) contained in the target gene sequence. Known amplification methods include, e.g., the polymerase chain reaction (PCR), transcription-associated amplification, replicase-mediated amplification, ligase chain reaction (LCR), Loop-mediated isothermal amplification (LAMP), Nucleic acid sequence-based amplification (NASBA) and strand-displacement amplification (SDA). Replicase-mediated amplification uses self-replicating RNA molecules, and a replicase such as QB-replicase (U.S. Pat. No. 4,786,600). PCR amplification uses DNA polymerase, primers and thermal cycling to synthesize multiple copies of two complementary strands of DNA or cDNA (U.S. Pat. Nos. 4,683,195, 4,683,202, and 4,800,159, and Methods in Enzymology, 1987, Vol. 155: 335-350). LCR amplification uses at least four separate oligonucleotides to amplify a target and its complementary strand by using multiple cycles of hybridization, ligation, and denaturation (e.g., U.S. Pat. No. 5,427,930, and U.S. Pat. No. 5,516,663). SDA uses a primer that contains a recognition site for a restriction endonuclease such that the endonuclease will nick one strand of a hemimodified DNA duplex that includes the target sequence, followed by amplification in a series of primer extension and strand displacement steps (e.g., U.S. Pat. No. 5,422,252, U.S. Pat. No. 5,547,861, U.S. Pat. No. 5,648,211). Loop-mediated isothermal amplification (LAMP) employs the self-recurring strand-displacement DNA synthesis primed by a specially designed set of the target-specific primers (Notomi T. et al., Nucleic Acids Research 2000; 28: e63). Nucleic acid sequence-based amplification (NASBA) is a primer-dependent technology that can be used for the continuous amplification of nucleic acids in a single mixture at one temperature (Compton J. et al., Nature 350 (6313), 91-92). It will be apparent to one skilled in the art that the oligonucleotides and methods illustrated by the preferred embodiments may be readily adapted to use in any primer-dependent amplification system by one skilled in the art of molecular biology (see Fred M. Ausubel, Roger Brent, Robert E. Kingston, David D. Moore, J. G. Seidman, John A. Smith, Kevin Struhl J., 2002. Current Protocols in Molecular Biology. John Wiley and Sons, New York and; Vadim V. Demidov, Natalia E. Broude, 2004. DNA Amplification: Current Technologies and Applications, Horizon Bioscience). Further, a number of reagents and systems to perform such amplification are commercially available.

In an embodiment, the amplification is performed using polymerase chain reaction (PCR). The PCR amplification step can be performed by standard techniques well known in the art (See, e.g., Sambrook, E. F. Fritsch, and T. Maniatis, Molecular Cloning: A Laboratory Manual, Second Edition, Cold Spring Harbor Laboratory Press (1989); U.S. Pat. No. 4,683,202; and PCR Protocols: A Guide to Methods and Applications, Innis et al., eds., Academic Press, Inc., San Diego (1990); Sambrook and Russell, Molecular Cloning: A Laboratory Manual, Third Edition, Cold Spring Harbor Laboratory Press (2000)). PCR cycling conditions typically consist of an initial denaturation step, which can be performed by heating the PCR reaction mixture to a temperature ranging from about 80° C. to about 105° C. for times ranging from about 1 to about 10 min. Heat denaturation is typically followed by a number of cycles, ranging from about 20 to about 50 cycles, each cycle usually comprising an initial denaturation step, followed by a primer annealing step and concluding with a primer extension step. Enzymatic extension of the primers by the nucleic acid polymerase, e.g. Taq polymerase, produces copies of the template that can be used as templates in subsequent cycles. An example of PCR conditions are: the reaction volume, in the range of 20-50 μl, preferably 50 μl, containing 0.1-100 fmols of the template in the presence of 0.5 to 2 μM, preferably 1 μM each of the primers, 100 μM each of dNTPs, 10 mM Tris-HCl, pH 8.3, 1.5 mM MgCl₂, 50 mM KCl, and 0.25 to 1 U, preferably 1U of Platinum™ Taq polymerase (Invitrogen, CA). Typically, 27-30 PCR cycles were used, preferably 27 cycles, consisting each of 30 s at 94° C., 30 s at 53° C. and 30 s at 72° C.

As used herein, the terms “discriminatory” or “discriminating” used in reference to the oligonucleotides of the present invention, means that the oligonucleotides are capable of selective binding to a first nucleic acid (i.e. a target or desired nucleic acid) relative to a second (undesired) nucleic acid. Similarly, the terms “detection” or “detecting” as used herein in reference to the methods using the oligonucleotides of the present invention means that the oligonucleotides are capable of selective binding to a first nucleic acid (i.e. a target or desired nucleic acid) relative to a second (undesired) nucleic acid. “Selective” as used herein, for example with respect to binding or hybridization, refers to a degree of binding/hybridization to a target (desired), which differs from a degree of binding to a non-target (undesired), and thus may be distinguished accordingly. For example, a greater degree of binding/hybridization to a target relative to a non-target allows for the detection of such selective binding/hybridization, which may be detected for example by virtue of a signal corresponding to target binding/hybridization which is greater than a lower signal corresponding to non-target binding/hybridization (i.e., a signal/noise ratio allowing detection). In such a case, such selective binding/hybridization to a target nucleic acid (sometimes referred to herein as a first nucleic acid) is indicative of the presence of the target nucleic acid (e.g., in a sample suspected of containing the target nucleic acid). Such selective binding/hybridization may be determined under a given set of conditions which may be determined by the skilled person for a given oligonucleotide and desired target (and undesired target) of interest. In embodiments, such selective binding/hybridization comprises binding/hybridization to a target (desired) nucleic acid that is at least 2-fold greater than binding/hybridization to a non-target (undesired) nucleic acid, in further embodiments at least 3, 4, 5, 6, 7, 8, 9 or 10-fold greater than binding/hybridization to a non-target nucleic acid.

As such, the methods of the invention allow for the detection of a target nucleic acid present in a given sample.

In an embodiment, the above-mentioned method further comprises selecting an oligonucleotide from said further amplified oligonucleotides on the basis of its preferential binding to said first nucleic acid relative to said second nucleic target.

“Hybridization” of nucleic acid sequences refers to the interaction or binding between nucleic acid sequences, for example on the basis of the complementary nature of the sequences. Hybridization may be performed under various conditions via the adjustment of various parameters therein. For example, hybridization may be performed under moderately stringent or stringent conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point (Tm), which corresponds to the temperature at which 50% of the oligonucleotide and its perfect complement are in duplex, or above, for the specific sequence at a defined ionic strength and pH.

Stringency of hybridization is related to Tm. When hybridization is carried out close to the Tm of perfectly base-paired duplexes, mismatched hybrids will not be stable. Such conditions, which prevent formation of duplexes of mismatched sequences are considered to be stringent or of high stringency. In contrast, conditions which favor the formation of mismatched duplexes are those considered as non-stringent or of low stringency, and may be effected typically by lowering the incubation temperature (see Andersen, Nucleic acid Hybridization, Springer, 1999, p. 54).

In an embodiment, the above-mentioned hybridization is performed at a temperature less than about 5° C. lower than the thermal melting point (Tm). In a further embodiment, the above-mentioned hybridization is performed at a temperature less than about 7° C. lower than the Tm. In a further embodiment, the above-mentioned hybridization is performed at a temperature less than about 10° C. lower than the Tm. In a further embodiment, the above-mentioned hybridization is performed at a temperature less than about 15° C. lower than the Tm. In an embodiment, the above-mentioned hybridization is performed at a temperature of about 50° C. or less. In a further embodiment, the above-mentioned hybridization is performed at a temperature between about 50° C. to about 4° C. In a further embodiment, the above-mentioned hybridization is performed at a temperature between about 15° C. to about 30° C. In a further embodiment, the above-mentioned hybridization is performed at a temperature between about 20° C. to about 28° C. (e.g., about 22° C. to about 25° C.), typically referred to as “room” or “ambient” temperature). In a further embodiment, the hybridization is performed in a buffer comprising about 10 mM Tris pH 7.0, 10 mM MgCl₂ and 500 mM NaCl.

The main factors affecting Tm are salt concentration, strand concentration, and the presence of denaturants (such as formamide or DMSO). Other effects such as sequence, length, and hybridization conditions can be important as well. Also, counter ion identity, solvation effects, conjugated groups (biotin, digoxigenin, alkaline phosphatase, fluorescent dyes, etc.), and impurities may also affect the Tm.

Various theoretical methods exist to calculate the Tm or the Td (the temperature at a particular salt concentration, and total strand concentration at which 50% of an oligonucleotide and its perfect filter-bound complement are in duplex) of a nucleic acid/oligonucleotide.

For example, Td can be calculated using the Wallace rule (Wallace, R. B. et al., Nucleic Acids Res. 6, 3543 (1979)):

Td=2° C.(A+T)+4° C.(G+C) (1)

Td is a filter-based calculation where A, G, C, and T are the number of occurrences of each nucleotide. This equation was developed for short DNA oligonucleotides of 14-20 base pairs hybridizing to membrane bound DNA targets in 0.9M NaCl.

The nature of the immobilized target strand provides a net decrease in the Tm observed when both target and probe are free in solution. The magnitude of the decrease is approximately 7-8° C.

Another familiar equation for DNA which is valid for oligonucleotides longer than 50 nucleotides from pH 5 to 9 is (Howley, P. M. et al., J. Biol. Chem. 254, 4876):

Tm=81.5+16.6 log M+41(XG+XC)−500/L−0.62F

where M is the molar concentration of monovalent cations, XG and XC are the mole fractions of G and C in the oligonucleotide, L is the length of the shortest strand in the duplex, and F is the molar concentration of formamide.

This equation includes adjustments for salt (although the equation is undefined when M=0) and formamide, the two most common agents for changing hybridization temperatures.

Another equation can be used to calculate the Tm using thermodynamic basis sets for nearest neighbor interactions (Breslauer, K. J. et al., Proc. Natl. Acad. Sci. USA 83, 3746-3750 (1986)). The equation is:

$$Tm = \frac{1000 ⋆ Δ H}{A + Δ S + R ln ( Ct / 4 ) - 273.15 + 16.6 log [ Na + ]}$$

where ΔH (Kcal/mol) is the sum of the nearest neighbor enthalpy changes for hybrids, A is a small, but important constant containing corrections for helix initiation, ΔS (eu) is the sum of the nearest neighbor entropy changes, R is the Gas Constant (1.987 cal deg-1 mol-1) and Ct is the total molar concentration of strands. If the strand is self-complementary, Ct/4 is replaced by Ct.

Therefore, stringency of hybridization may be controlled to favor the formation of mismatched duplexes.

Similarly, washing of hybridized samples may be performed under conditions which also maintain the interactions of mismatched duplexes.

In a further embodiment, the removing (or washing) step mentioned herein is performed under the same or lower stringency conditions than the hybridizing step. In an embodiment, the above-mentioned washing is performed at a temperature less than about 5° C. lower than the thermal melting point (Tm). In a further embodiment, the above-mentioned washing is performed at a temperature less than about 7° C. lower than the Tm. In a further embodiment, the above-mentioned washing is performed at a temperature less than about 10° C. lower than the Tm. In a further embodiment, the above-mentioned washing is performed at a temperature less than about 15° C. lower than the Tm. In an embodiment, the above-mentioned washing is performed at a temperature of about 50° C. or less. In a further embodiment, the above-mentioned washing is performed at a temperature between about 50° C. to about 4° C. In a further embodiment, the above-mentioned washing is performed at a temperature between about 15° C. to about 30° C. In a further embodiment, the above-mentioned washing is performed at a temperature between about 20° C. to about 28° C. (e.g., about 22° C. to about 25° C.), typically referred to as “room” or “ambient” temperature).

In an embodiment, the above-mentioned dissociation (step (c)) is performed by incubation at an elevated temperature relative to said hybridization. In an embodiment, the above-mentioned temperature is a temperature above the melting temperature (Tm). In a further embodiment, the above-mentioned elevated temperature is at least about 2° C. above the Tm. In a further embodiment, the above-mentioned elevated temperature is at least about 5° C. above the Tm. In a further embodiment, the above-mentioned elevated temperature is at least about 10° C. above the Tm. In a further embodiment, the above-mentioned elevated temperature is at least about 15° C. above the Tm. In a further embodiment, the above-mentioned elevated temperature is at least about 85° C.

The invention further provides the above-mentioned method wherein said hybridization is performed in the presence of a blocking agent capable of inhibiting binding of said primer recognition sequences to said first target nucleic acid. In an embodiment, said blocking agent is an oligonucleotide capable of binding said primer recognition sequences (e.g., an oligonucleotide complementary or substantially complementary to the primer recognition sequences).

The invention further provides the above-mentioned method, wherein the desired nucleic acid is derived from a pathogen. In an embodiment, said pathogen is selected from a eukaryote, prokaryote and a virus. In a further embodiment, said virus is human papillomavirus (HPV) and said first and second nucleic acids are derived from different subtypes of HPV. In an embodiment, said subtypes are selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

In embodiments, the methods of the invention may be carried out on a solid support, i.e. having one or more reagents bound to the solid support. Solid supports may be comprised of any material including but not limited to conducting materials, semiconducting materials, thermoelectric materials, magnetic materials, light-emitting materials, biominerals and polymers. Non-limiting examples of solid substrates are a microtiter plate, a membrane, a microsphere (bead) or a chip.

The conducting material may be a metal, such as a transition metal. Examples of transition metals include, but are not limited to silver, gold, copper, platinum, nickel and palladium.

Examples of semiconducting materials that may be used as solid supports include, but are not limited to a group IV semiconducting material, a group II-VI semiconducting material and a group III-V semiconducting material. As used herein, the term “Group” is given its usual definition as understood by one of ordinary skill in the art. For instance, Group II elements include Zn, Cd and Hg; Group III elements include B, Al, Ga, In and Tl; Group IV elements include C, Si, Ge, Sn and Pb; Group V elements include N, P, As, Sb and Bi; and Group VI elements include O, S, Se, Te and Po.

The magnetic material may be any magnetic material such as a paramagnetic material or a ferromagnetic material. Examples of paramagnetic materials that can be used according to this aspect of the present invention include, but are not limited to aluminum, copper, and platinum. Examples of ferromagnetic materials that can be used according to this aspect of the present invention include, but are not limited to magnetite, cobalt, nickel and iron.

Examples of light-emitting materials that may be used according to this aspect of the present invention include, but are not limited to dysprosium, europium, terbium, ruthenium, thulium, neodymium, erbium, ytterbium and any organic complex thereof.

An example of a biomineral that may be used according to this aspect of the present invention is calcium carbonate.

Examples of polymers that may be used according to this aspect of the present invention include, but are not limited to polyethylene, polystyrene and polyvinyl chloride.

Examples of thermoelectric materials that may be used according to this aspect of the present invention include, but are not limited to bismuth telluride, bismuth selenide, bismuth antimony telluride and bismuth selenium telluride.

Various equipment and means to confer temperature control and reagents and means to confer the concentration of salts, additional factors, pH and reaction conditions (e.g., suitable buffers) are known in the art and may be used in the methods of the invention.

The invention further provides the above-mentioned method, wherein said first and second nucleic acids differ by at least 1 nucleotide, in a further embodiment, at least 2 nucleotides, in a further embodiment, at least 3 nucleotides, in further embodiments, at least 4, 5, 6, 7, 8, 9 or 10 nucleotides.

The invention further provides the above-mentioned method, wherein the random nucleotide sequence of said further amplified oligonucleotides is not exactly complementary to said first nucleic acid. In an embodiment, the random nucleotide sequence of said further amplified oligonucleotides comprises at least 1 mismatch, in a further embodiment, at least 2 mismatches, in a further embodiment, at least 3 mismatches relative to said first nucleic acid. In an embodiment, the random nucleotide sequence of said further amplified oligonucleotides comprises 1 to 10 mismatches relative to said first nucleic acid.

In an embodiment, the invention provides the above-mentioned method, wherein said first nucleic acid is single-stranded and said amplified oligonucleotides are treated, prior to further hybridization, to degrade/remove the strand of said amplified oligonucleotides which is not hybridizing (i.e. which is not partially or fully complementary) to said single-stranded first nucleic acid. In an embodiment, said treatment is with an exonuclease capable of selective degradation of said strand of said amplified oligonucleotides which is not hybridizing (i.e. which is not partially or fully complementary) to said single-stranded first nucleic acid. In embodiments, said selectivity is based on 5′-terminal phosphorylation of said strand and said exonuclease is lambda (λ) exonuclease.

In another aspect, the present invention provides a kit for identifying an oligonucleotide for discriminating a first nucleic acid from a second nucleic acid, the kit comprising for example the above-mentioned pool of oligonucleotides. In embodiments, the kit further comprises instructions setting forth the above-mentioned method for identifying an oligonucleotide for discriminating a first nucleic acid from a second nucleic acid. In further embodiments, the kit further comprises the above-mentioned first nucleic acid and/or second nucleic acid. In further embodiments, the kit comprises the above-mentioned primers which correspond to the above-mentioned primer recognition sequences. In further embodiments, the kit comprises the above-mentioned blocking agent (e.g., an oligonucleotide capable of binding the primer recognition sequences [e.g., an oligonucleotide partially or fully complementary to the primer recognition sequences]). In further embodiments, the kit further comprises one or more suitable reagents (e.g. buffers/solutions/factors/components/reagents suitable for hybridization, washes, amplification and/or detection) to facilitate or effect hybridization, amplification and/or detection, e.g., to provide suitable factors or components and/or to regulate pH and/or ionic strength.

In another aspect, the present invention provides an oligonucleotide obtained by the above-mentioned method.

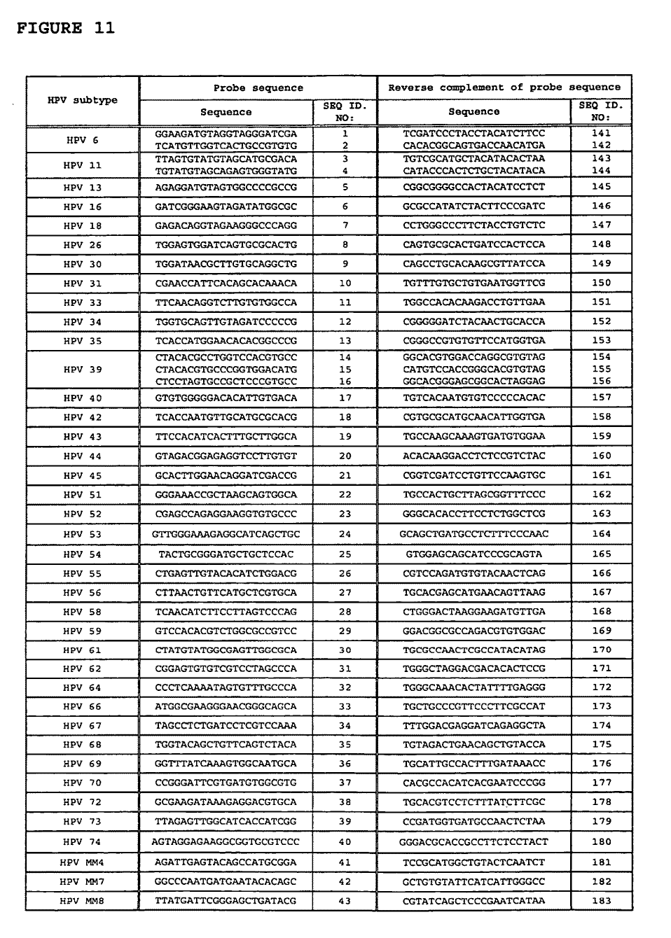

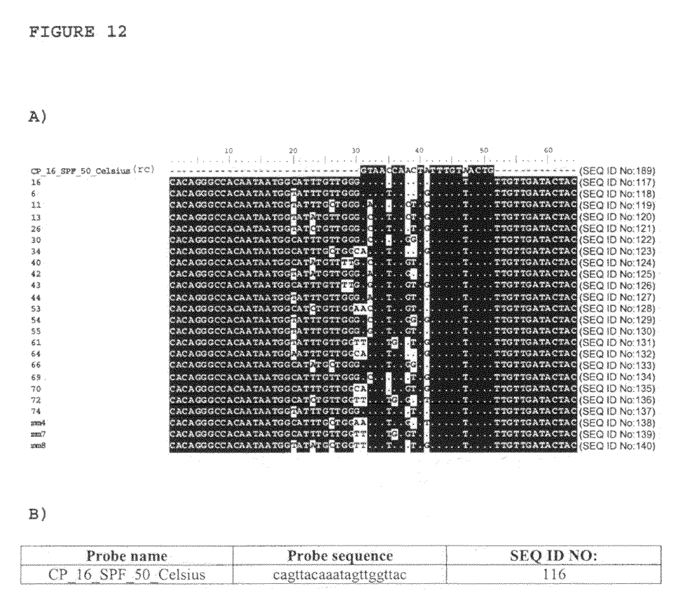

In another aspect, the present invention provides an oligonucleotide capable of discriminating a first nucleic acid from a second nucleic acid (e.g., when used as a probe or a primer), wherein said oligonucleotide is not exactly complementary to said first nucleic acid. In an embodiment, said oligonucleotide comprises at least at least 1 mismatch, in a further embodiment, at least 2 mismatches, in a further embodiment, at least 3 mismatches relative to said first nucleic acid. In an embodiment, the oligonucleotide comprises 1 to 10 mismatches relative to said first nucleic acid. In an embodiment, said first nucleic acid is derived from a pathogen. In a further embodiment, said pathogen is selected from a eukaryote, prokaryote and a virus. In a further embodiment, said virus is human papillomavirus (HPV). In a further embodiment, said first and second nucleic acids are derived from different subtypes of HPV. In an embodiment, said subtypes are selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8. In a further embodiment, said oligonucleotide comprises a nucleotide sequence selected from SEQ ID NOs: 1-43, 100-104 and 116, or a complement thereof. In a further embodiment, said oligonucleotide comprises a sequence and is capable of selectively detecting an HPV subtype as set forth in FIG. 11.

In another aspect, the present invention provides a collection of two or more oligonucleotides of the invention. In an embodiment, the above-mentioned oligonucleotides comprise a nucleotide sequence selected from SEQ ID NOs: 1-43, 100-104 and 116, or a complement thereof. In an embodiment, the above-mentioned oligonucleotides are immobilized on a substrate. In another embodiment, the oligonucleotides are labelled with a detectable marker. In a further embodiment, the above-mentioned detectable marker is a fluorescent moiety. In another embodiment, the above-mentioned oligonucleotides are hybridizable array elements in an array (e.g, a microarray).

In another aspect, the present invention provides a method for detecting the presence of a first nucleic acid in a sample, said method comprising contacting the above-mentioned oligonucleotide with said sample under conditions permitting selective hybridization of said oligonucleotide to said first nucleic acid, wherein selective hybridization is indicative that said first nucleic acid is present in said sample. In an embodiment, said first nucleic acid is derived from a pathogen and said method is for detection of said pathogen in a sample. In an embodiment, said oligonucleotide is bound to a solid support (e.g, an array). In an embodiment, said sample is a biological sample derived from a subject and said method is for detection of said pathogen in said subject. In an embodiment, said method is for diagnosing a disease or condition associated with said pathogen in said subject. In a further embodiment, said pathogen is selected from a eukaryote, prokaryote and a virus. In a further embodiment, said virus is human papillomavirus (HPV). In an embodiment, the above-mentioned disease or condition is cancer (e.g., cervical cancer). In a further embodiment, said first and second nucleic acids are derived from different subtypes of HPV. In an embodiment, said subtypes are selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8. In an embodiment, said subject is a mammal. In a further embodiment, said mammal is a human.

In various embodiments, the above-mentioned method may further comprise extraction, isolation, modification and/or amplification (or other such treatments) of nucleic acid preparations from said sample, e.g., prior to contacting with an oligonucleotide of the invention.

In various embodiments, the above-mentioned oligonucleotide or first nucleic acid may be bound to a solid support (e.g. an array) or be present in a free form in solution. In another embodiment, the above-mentioned oligonucleotide or first nucleic acid may be labelled with a detectable marker (e.g., a fluorescent marker) such that the presence or amount of the nucleic acid or oligonucleotide can be detected by assessing the presence/level of the label.

As used herein, a “biological sample” refers to any tissue or material derived from a living or dead organism which may contain the target nucleic acid, including, in the case of an animal for example, samples of blood, urine, semen, milk, sputum, mucus, pleural fluid, pelvic fluid, synovial fluid, ascites fluid, body cavity washes, eye brushing, skin scrapings, a buccal swab, a vaginal swab, a pap smear, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, a microbial culture, a virus, and samples of in vitro cell culture constituents. A biological sample may be treated to physically or mechanically disrupt tissue or cell structure to release intracellular components into a solution which may further contain enzymes, buffers, salts, detergents and the like, using well known methods. Cell samples may be obtained from a subject by a variety of techniques including, for example, by scraping or swabbing an area, or by using a needle to biopsy solid tumors or to aspirate body fluids from the chest cavity, bladder, spinal canal, or other appropriate area.

In another aspect, the present invention provides a kit for detecting the presence of a first nucleic acid in a sample, said kit comprising the above-mentioned mentioned oligonucleotide or collection of oligonucleotides. In a further embodiment, said kit comprises:
(a) the above-mentioned oligonucleotide or collection of oligonucleotides; and
(b) means for detecting selective hybridization of said oligonucleotide(s) to said first nucleic acid.

Such “means for detecting” may in various embodiments comprise a suitable labelling system, such as for example the labelling systems noted above. Such kits may further comprise one or more suitable reagents (e.g. buffers/solutions/factors/components suitable for hybridization, washes, amplification and/or detection) to facilitate or effect hybridization, amplification and/or detection, e.g., to provide suitable factors or components and/or to regulate pH and/or ionic strength.

The oligonucleotides and e.g., reagents of the kit may be provided in various formats. For example, the oligonucleotides may be provided in a free form or bound to a suitable substrate.

In an embodiment, the above-mentioned kit further comprises instructions setting forth the above-mentioned method. In a further embodiment, said first nucleic acid is derived from a pathogen and said kit is for detecting the presence of said pathogen in said sample. In a further embodiment, said sample is a biological sample derived from a subject and said kit is for detection of said pathogen in said subject. In an embodiment, said kit is for diagnosing a disease or condition associated with said pathogen in said subject. In a further embodiment, said pathogen is selected from a eukaryote, prokaryote and a virus. In a further embodiment, said virus is human papillomavirus (HPV). In a further embodiment, said first and second nucleic acids are derived from different subtypes of HPV. In an embodiment, said subtypes are selected from HPV 6, HPV 11, HPV 13, HPV 16, HPV 18, HPV 26, HPV 30, HPV 31, HPV 33, HPV 34, HPV 35, HPV 40, HPV 42, HPV 43, HPV 44, HPV 45, HPV 51, HPV 52, HPV 53, HPV 54, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

In an embodiment, the kit may comprise a plurality (e.g. a collection) of the above-mentioned oligonucleotides thereby to allow the identification of a plurality of different nucleic acids of interest, which for example may correspond to different pathogens of interest and thus allow the identification of a plurality of pathogens.

The oligonucleotides, methods and kits of the invention may for example be used in analytical, diagnostic (e.g., infection of an animal, plant or organism [e.g., a cell or tissue culture] by a pathogen), detection, manufacturing/quality control, research, environmental monitoring (e.g., pollution/contamination of air/water/reagents intended for use in biological systems (e.g. culture or animal systems)/other materials), microbiology (detection; studies of non- or difficult to cultivate organisms) and forensic applications, as well as others.

In another aspect, the present invention provides an array comprising the above-mentioned oligonucleotide or the above-mentioned collection of two or more oligonucleotides.

The term “array” encompasses the term “microarray” and refers to an ordered array presented for binding to nucleic acids and the like. An “array,” includes any two-dimensional or substantially two-dimensional (as well as a three-dimensional) arrangement of spatially addressable regions bearing nucleic acids, particularly oligonucleotides or synthetic mimetics thereof, and the like, e.g., UNA oligonucleotides. Where the arrays are arrays of nucleic acids, the nucleic acids may be adsorbed, physisorbed, chemisorbed, or covalently attached to the arrays at any point or points along the nucleic acid chain. Methods for the preparation of nucleic acid arrays, particularly oligonucleotide arrays, are well known in the art (see, e.g., Harrington et al., Curr Opin Microbiol. (2000) 3:285-91, and Lipshutz et al., Nat. Genet. (1999) 21:20-4). The subject nucleic acid arrays can be fabricated using any means available, including drop deposition from pulse jets or from fluid-filled tips, etc, or using photolithographic means. Either polynucleotide precursor units (such as nucleotide monomers), in the case of in situ fabrication, or previously synthesized polynucleotides can be deposited. Such methods are described in detail in, for example U.S. Pat. Nos. 6,242,266, 6,232,072, 6,180,351, 6,171,797, and 6,323,043.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

The present invention is illustrated in further details by the following non-limiting examples.

### Example 1

### Generation of Oligonucleotide Probes to Discriminate Between Closely Related DNA Sequences

### Materials and Methods

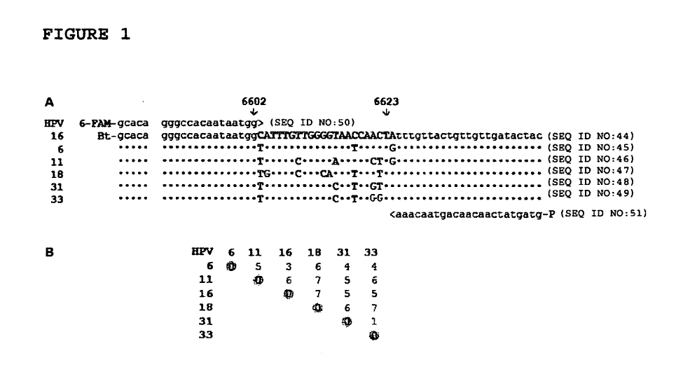

Oligonucleotides. All oligonucleotides were synthesized by Integrated DNA Technologies (Coralville, Iowa). Target oligonucleotides corresponding to the so-called short PCR fragment, SPF, described by Kleter et al. (Kleter et al. 1999, J Clin Microbiol 37, 2508-17), consisted of 22-nucleotide long, HPV type-specific segment, flanked by 20 and 23-nucleotide long PCR primers anchoring sequences as illustrated in FIG. 1 (SEQ ID NOs: 44-49). These 65-nucleotide long oligomers were synthesized in two versions: non-modified and modified at their 5′ ends with biotin to allow for their immobilization on streptavidin-coated solid supports. The corresponding forward and reverse primers (SEQ ID NOs: 50 and 51) were used to amplify the synthetic targets or the corresponding HPV DNAs obtained from the clinical samples; these primers were modified at their 5′ ends by addition of 6-carboxyflorescein, 6-FAM, and the phosphate residue, respectively.

Oligonucleotide probes were obtained by rounds of hybridizations starting with mixture containing 22 nucleotide long random sequence segment embedded within constant sequence fragments to anchor PCR primers, ROM22: GCCTGTTGTGAGCCTCCTGTCGAA-(N)22-TTGAGCGTTTATTCTTGTCTCCCA (SEQ ID NO: 52), where “N” corresponds to A, G, C and T (equimolar during synthesis). The following oligonucleotides were used to block the flanking primer-anchoring segments of ROM22: 5′ blocker, TTCGACAGGAGGCTCACAACAGGC (SEQ ID NO: 53) and 3′ blocker, 5′P-TGGGAGACAAGAATAAACGCTCAA (SEQ ID NO: 54). The oligonucleotide GCCTGTTGTGAGCCTCCTGTCGAA (SEQ ID NO: 55), complementary to the 5′ blocker, was used as the forward primer and the 5′-phosphorylated 3′ blocker (SEQ ID NO: 54) as the reverse primer, serving in PCR to amplify (i) pools of oligonucleotide mixtures (pooled probes PP) obtained after each cycle of hybridization, or (ii) particular probes (cloned probes CP) from the plasmid clones carrying individual oligonucleotide sequences. Finally, target complements represented 22-nucleotides long complementary sequences of the HPV type-specific SPF segments listed in FIG. 1, all modified at 5′ end by the addition of 6-FAM.

Clinical Samples. DNA was extracted from six patients containing single type HPV. Initially, DNA was amplified with PGMY primers (Gravitt et al. 2000, J Clin Microbiol. 38, 357-61) and typed by sequencing.

Immobilization of target oligonucleotides. Streptavidin-coated tubes (Roche Diagnostics GmbH, Mannheim, Germany) and 96-well plates (Pierce Reacti-Bind Streptavidin Coated High Binding Capacity Black plates, Rockford, Il) were used for preparative and analytical purposes, respectively. After washing 3 times with 10 mM Tris-HCl, pH 7.5, 10 mM MgCl₂ and 50 mM NaCl (TMN buffer), the tubes (or plates) were incubated with the predefined amount, between 1 and 100 pmoles, of the 5′ biotinylated target oligonucleotide for at least 15 minutes, rinsed 3 times with TMN buffer, and stored at 4° C. until used.

Amplification and conversion of oligonucleotides into single stranded DNA. Following hybridization, the bound oligonucleotides were dissociated from the target. These PP were amplified by PCR: the reaction was carried out in a total volume of 50 μl containing 0.1-100 fmols of the template in the presence of 1 μM each of the primers (FIG. 1), 100 mM each of dNTPs, 10 mM Tris-HCl, pH 8.3, 1.5 mM MgCl₂, 50 mM KCl, and 1 U of Platinum™ Taq polymerase (Invitrogen, CA). Typically, 27-30 PCR cycles were used, consisting each of 30 s at 94° C., 30 s at 53° C. and 30 s at 72° C. The quantity and quality of PCR products were estimated by agarose gel-electrophoresis and/or by measuring the 6-FAM fluorescence of PCR amplicons, after eliminating the non-incorporated primers, using the Montage centrifuge filter device (Millipore, Billerica, Mass.). These products were rendered single stranded by incubation with 5 U of λ exonuclease (NEB, Boston, Mass.) that digests 5′-phosphorylated strand, for 30 min at 37° C., followed by 20 minutes at 65° C. to inactivate the enzyme. The same procedure was used to produce single stranded probes from PCR products from individual clones.

Hybridizations. The synthetic mixture of random oligonucleotides ROM22 (1 nmole) was used in the initial hybridization cycle to obtain the first generation of PP. In all subsequent hybridizations, the PPs from the preceding cycle were PCR amplified and converted to the single stranded form. Typically, 10-50 pmoles of single stranded PP (0.05-0.25 μM) obtained in the previous cycle was mixed with two blocking oligonucleotides to obtain 0.5 μM each, in 200 ml of TMN buffer and heated to 90° C. This solution was subsequently transferred to tubes containing prebound biotinylated targets, then cooled down to the ambient temperature, 22-24° C., and left for at least 4 hours at this temperature. The tubes were then rinsed 3 times with TMN buffer and the probes that remained bound to the targets were washed off by incubation at 90° C. in 200 ml of water for 2 min. There was 1 pmole of the added target per tube, except during the first hybridization when 100 pmoles were added (however, the effective amount of the available target for binding was less, see below). Positive hybridizations above were followed by subtractive hybridizations carried as above but in the presence of 0.5 μM (total) of the non-desired oligonucleotide targets (i.e. other than the immobilized target).

Binding Experiments. Target oligonucleotides, representing SPF of different HPV types, were immobilized in separate wells of 96-well plates (under saturation with target, the resulting effective amount of the target per well was about 17 pmoles, when measured as its amount available for binding with its 6-FAM-labelled complement). PP or CP (0.1-0.5 μM, converted to single strands) were incubated with immobilized targets, in the presence of 1 μM each of the block oligonucleotides, in 100 ml of TMN buffer for 4 hours at 22° C. The wells were rinsed 3 times with 100 ml of TMN buffer and the bound 6-FAM fluorescence (in relative fluorescence units, RFU) was measured directly in Spectra MAX Gemini XS (22° C., lex=485 nm and lem=538 nm). The binding experiments with the 6-FAM labelled, 22-nucleotides long target complements were carried out using the same protocol, except that blockers were not added.

Competitive Binding. The binding was measured as above, with 6-FAM labelled oligonucleotides (PP, CP or complements) kept at constant concentration of 10-50 pmoles/well (0.1-0.5 μM), in the presence of the increasing concentrations, from zero to 10 μM, of target competitor. The latter was the non-biotinylated SPF oligonucleotide, either identical with the immobilized target (homologous competitive binding), or representing the SPF sequence of another HPV type (heterologous competitive binding). The EC₅₀ values were estimated form the data according to the equation calculated from using the GraphPad Prism™ Software (Version 4).

Cloning and sequencing of individual probes. Cloning the probes from the PPs was done using TOPO TA Cloning™ kit (Invitrogen, CA). Typically, twenty positive clones were selected using X-Gal/IPTG based-colorimetric reaction, following the manufacturer's protocol. The M13 forward and reverse primers were used to confirm the presence of the insert and to “extract” it for subsequent direct sequence determination using LiCor apparatus (Lincoln, Nebr.). In turn, the resulting CPs were produced by PCR using ROM22 primers and tested for binding.

Reverse format hybridization. The sequences of the cloned probes with the best signal to noise ratio were chemically synthesized (IDT) with a biotin moiety at their 5′ end. Individual 5′ biotinylated probes were bound (100 pmoles) to streptavidin-coated plates. The HPV SPF were generated by PCR either from the typed DNA obtained from clinical samples or from the synthetic target oligonucleotides (FIG. 1), using 0.1 fmole of the template and the corresponding 6-FAM-labelled and 5′-phosphorylated forward and reverse primers (0.15 μM of each), following Kleter's procedure (Kleter et al., 1999, J Clin Microbiol 37, 2508-17). The reaction was carried out in 50 μl in the presence of 100 μM of each of dNTPs, 10 mM Tris-HCl (pH 8.3), 1.5 mM MgCl₂, 50 mM KCl, and 1 U of Platinum™ Taq polymerase (Invitrogen, CA), for 40 cycles, consisting of 30 s incubation at 94° C., 30 s at 52° C. and 30 s at 72° C. The PCR products (10-30 pmols) were converted to single stranded DNA and mixed with 200 pmoles of each of the blockers (two-fold excess over the added immobilized probe). Prior to transferring into the micro titer well, this mixture was heated to 90° C. and the hybridization was performed overnight or for at least 4 hours at ambient temperature. The wells were washed three times with TMN buffer and the fluorescence was directly measured in Spectra MAX Gemini XS and at 22° C. as described.

### Results

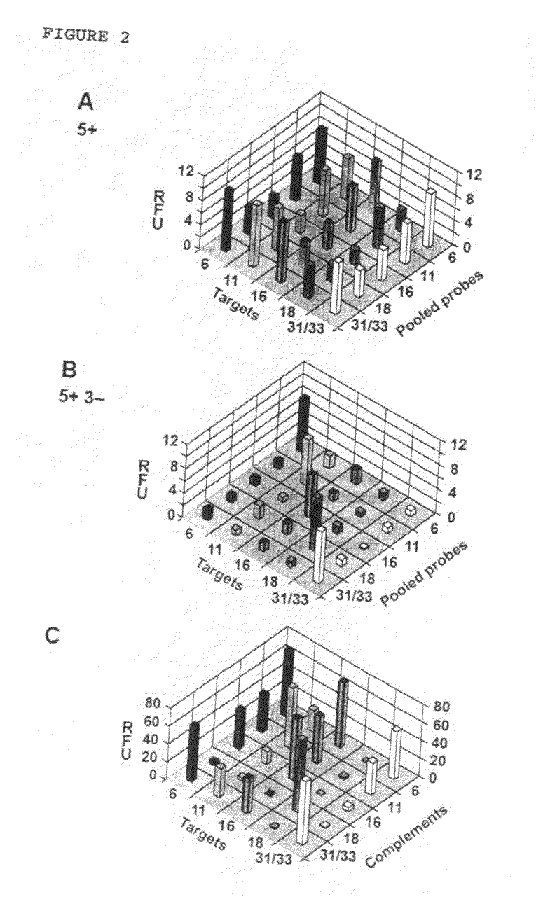

In the studies described herein, a series of iterative hybridizations were carried out to select probes recognizing six sequence variants of the “short HPV PCR fragment”, SPF (Kleter et al. 1999, supra). SPF targets consisted of 22-nucleotide long amplified portion flanked by 20-nucleotide and 23-nucleotide long primer sequences (FIG. 1A). They represented different HPV subtypes 6, 11, 16, 18, 31 and 33, differing by 3 to 7 nucleotides within the amplified portion (FIG. 1B) with types 31 and 33 differing only by one nucleotide position that eventually will be considered together. Synthetic, biotinylated target oligonucleotides were immobilized in the streptavidin coated tubes and were hybridized to a mixture of synthetic random oligonucleotides, ROM22, consisting of 22-nucleotide random sequence flanked by two 24-nucleotide long primer sequences. Following the first hybridization, the unbound ROM22 oligonucleotides were washed away and the bound ones were dissociated from their targets, re-amplified by PCR and hybridized again. Each hybridization cycle enriched the resulting mixture of pooled probes in sequences that were efficiently binding their targets. Yet, as can be seen in FIG. 2A, some of these pooled probes (PPs) obtained after five cycles of iterative hybridizations (5+), bind their corresponding cognate targets. As shown in FIG. 2B, the specificity of the resulting PP was improved after they were submitted to three additional cycles of the subtractive hybridization, i.e. in the presence of mixture of undesired targets (5+3−). The intensity of the specific signal (diagonal) remained the same, whereas the non-specific hybridization was decreased, to the background level at several instances. Thus, the performance of PP submitted to the process of iterative hybridization that includes subtractive (−) cycles largely surpass that of the PP obtained when this process consisted only of the forward (+) hybridization cycles. As shown in FIG. 2, PPs at the end of 5+3− cycles also perform much better than the 22-nucleotide long complements of the analyzed targets. These complements when used as probes readily cross-hybridize with the mismatched non-cognate targets (FIG. 2C).

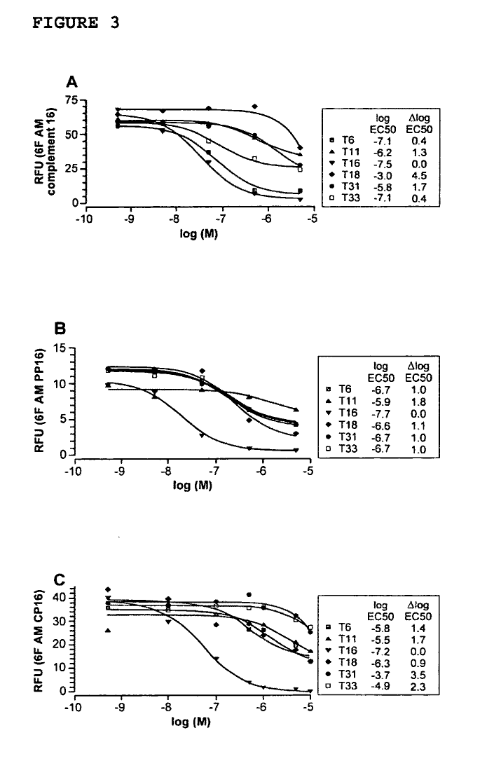

The capacity of discrimination of a probe between different targets can be studied by competitive hybridization in which the extent of the probe:target complex is measured at varying concentrations of the competitor. If the target is immobilized and the probe is labelled one may titrate the complex by increasing the concentration of the free targets. The effective concentration required to dissociate 50% of the original complex, EC₅₀, provides a measure of the competitor binding. The difference between EC₅₀ for the cognate oligonucleotide target and the EC₅₀ estimates for the non-cognate oligonucleotide targets provides the measure of the discrimination capacity of the probe. FIG. 3 illustrates the titration experiment carried with the immobilized HPV16 variant and its cognate probes. In FIG. 3A, the complement 16 was used as a probe. It discriminates very well against target HPV18 (T18). Yet, in the same time, it shows log EC₅₀ difference between the cognate T16 and T6 of only 0.4, indicating very poor discrimination. This can also be directly appreciated by looking at the corresponding titration curves that almost overlap (FIG. 3A) and the binding results presented at FIG. 2C. In contrast, PP16 shown in FIG. 3B discriminates similarly between cognate T16 and other targets with log EC₅₀ difference of 1.0 or more. Here T18 and T6 compete with the cognate T16:PP16 complex very similarly, in spite of the fact that the first differ from T16 by 7 and the second by only 3 nucleotide positions (FIG. 1B). Therefore, PP16 reveals desired characteristics of a probe that similarly discriminates multiple targets. It was chosen to be shown here since its cognate target differs by only 3 nucleotides from the closest HPV6 sequence.

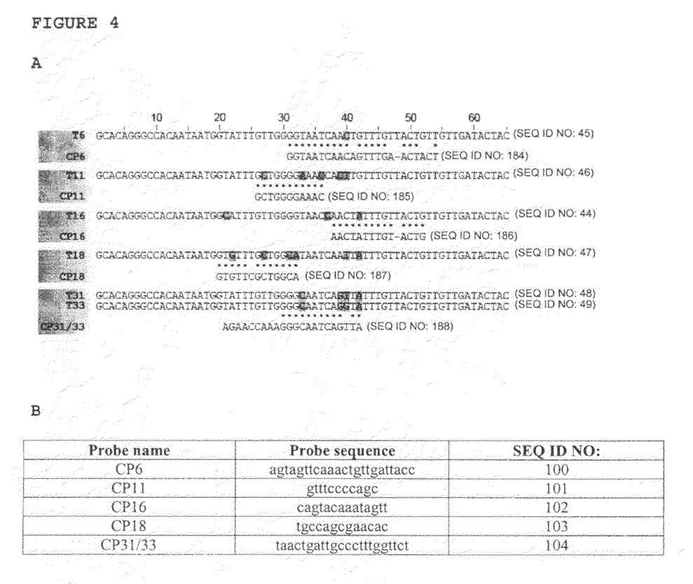

Each of the specific PP, following 5+3− cycles of iterative hybridization described above, consists of a mixture of different sequences. The corresponding unique sequence probes, CP (for Cloned Probe), were obtained by cloning PPs into plasmid vector. Individual CPs were extracted from the obtained plasmids by PCR and tested for binding to the cognate and non-cognate targets. It usually took less than 5 clones, to obtain one with the desired, arbitrarily defined ratio of at least 5 to one of the specific to non-specific binding. CPs that were retained for further analysis are shown in FIG. 4, where they are compared to their cognate targets.

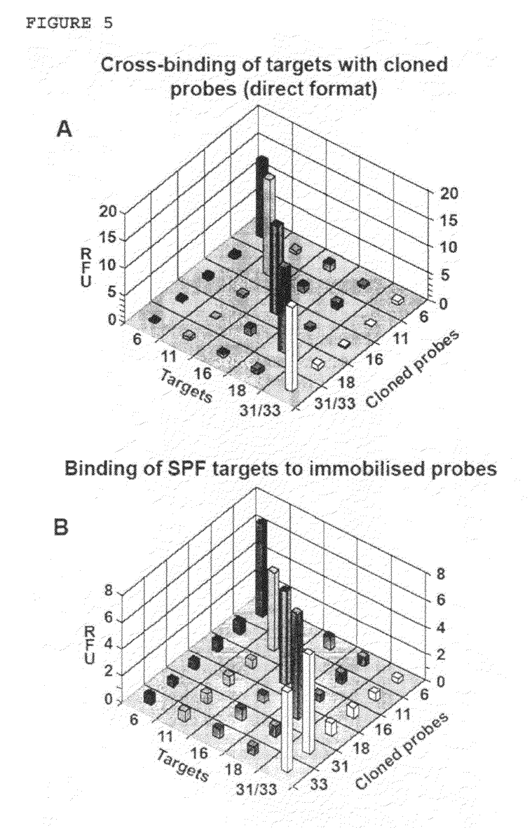

CPs performed better than PPs as far as the detection of their cognate targets and discrimination against the non-cognate ones is concerned (FIG. 5A). In a competitive titration shown in FIG. 3C, CP16 performed on average also better than its maternal PP16 (FIG. 3B) as judged by differences in log EC₅₀ values between the cognate T16 oligonucleotide and the non-cognate competitors. In other words, the latter were less efficient in chasing CP16 from the complex with T16 than in the case of PP16 in FIG. 3B. Finally, in binding experiments including all targets (FIG. 5A), CPs gave the same hybridization signal as their corresponding PPs (FIG. 2B), but less background hybridization. Furthermore, the advantage of CPs over their maternal PPs is that they may be used as tools in diagnostic tests that require hybridization in the reverse configuration, with probes immobilized to the solid support. Indeed, all the experiments reported so far were in the “forward blot format” with the immobilized targets. In the “reverse blot format” the probes, with biotin moiety at their 5′ end, are themselves immobilized and therefore can provide a simultaneous test for the presence of different targets, such as nucleic acids from distinct HPV variants in a clinical sample. This corresponds to the diagnostic situation where the target sequence amplified from a clinical sample is being tested in a panel of immobilized probes intended to positively identify the presence of a specific HPV subtype. As shown in FIG. 5B, CPs perform very well in the reverse blot format. Similar results were obtained when clinical samples of known HPV type were used as a source of the HPV SPF segment tested.

### Example 2

### Hybridization Probes for 39 Different Types of Human Papillomaviruses

### Materials and Methods

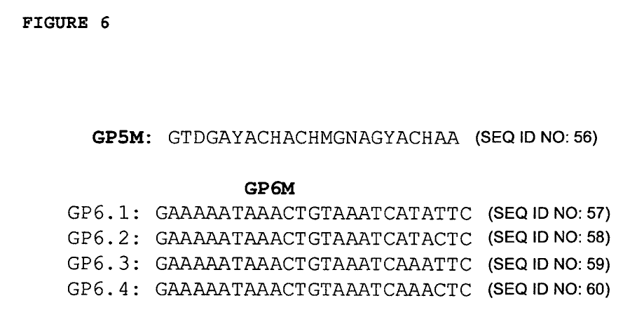

Oligonucleotides. All oligonucleotides were synthesized by Integrated DNA Technologies (IDT, Coralville, Iowa). Target oligonucleotides (SEQ ID NOs: 61-99), corresponding to 91-100 nucleotides long type-specific segments, originating from L1 HPV region, located between nucleotides 6647 and 6740, where HPV16 complete genome was used as a reference DNA (GenBank accession number K02718, GI:333031), (Seedorf, K. et al., 1985, Virology 145: 181-185). This region is flanked by 23 nucleotides-long forward and 24 nucleotides-long reverse universal PCR primers anchoring sequences, as illustrated in FIG. 6. The forward primer GP5M (SEQ ID NO: 56), with eight degenerative positions was designed to satisfy full-match priming requirements for all viral types (GP5M: GTDGAYACHACHMGNAGYACHAA) and its overlap with the binding site of GP5+ (Van den Brule et al., 2002, J Clin Microbiol 40, 779-87). The mixture of four reverse primers (GP6.1-GP6.4) is binding to GP6+ primer-binding site (Van den Brule et al., 2002, supra), but follows full-match priming requirements at the first five positions of 3′ end, for all 39 HPV types. The nucleotide sequences are as follows: GP6.1 (SEQ ID NO: 57), GAAAAATAAACTGTAAATCATATTC, GP6.2 (SEQ ID NO: 58), GAAAAATAAACTGTAAATCATACTC, GP6.3 (SEQ ID NO: 59), GAAAAATAAACTGTAAATCAAATTC and GP6.4 (SEQ ID NO: 60): GAAAAATAAACTGTAAATCAAACTC. Targets, presenting GP5+/6+ amplicons without forward and reverse primers sequences, were synthesized in two versions: non-modified and modified at their 5′ ends with biotin to allow for their immobilization on streptavidin-coated solid supports. Probe oligonucleotides were obtained by rounds of hybridizations, starting with a mixture containing a 22 nucleotides-long random sequence segment, ROM22: GCCTGTTGTGAGCCTCCTGTCGAA-(N)₂₂-TTGAGCGTTTATTCTTGTCTCCCA (SEQ ID NO: 52), where N-corresponds to A, G, C and T (equimolar during synthesis), embedded within constant sequence fragments to anchor PCR primers. The following oligonucleotides were used to block the flanking primer anchoring segments of ROM22: 5′ block, TTCGACAGGAGGCTCACAACAGGC (SEQ ID NO: 53) and 3′ block, 5′P-TGGGAGACAAGAATAAACGCTCAA (SEQ ID NO: 54). The oligonucleotide GCCTGTTGTGAGCCTCCTGTCGAA (SEQ ID NO: 55), complementary to the 5′ block, was used as the forward primer and the 5′-phosphorylated 3′ block oligonucleotide as the reverse primer, serving to PCR amplify (i) the target-specific oligonucleotide mixtures, called pooled probes (PP) obtained after each cycle of hybridization, or (ii) the particular probes from the plasmid clones, called cloned probes (CP), carrying individual oligonucleotide sequences.

Immobilization of target oligonucleotides. Streptavidin-coated tubes (Roche Diagnostics GmbH, Mannheim, Germany) and 96-well plates (Pierce Reacti-Bind Streptavidin Coated High Binding Capacity Black plates, Rockford, Il) were used for preparative and analytical purposes, respectively. After washing 3 times with 10 mM Tris-HCl, pH 7.5, 10 mM MgCl₂ and 50 mM NaCl (TMN buffer), the tubes (or plates) were incubated with the predefined amount, between 1 and 100 pmoles, of the 5′ biotinylated target oligonucleotide for at least 15 minutes, rinsed 3 times with TMN buffer, and stored at 4° C. until use.

Amplification and conversion of oligonucleotides into single stranded DNA. Following hybridization, the bound oligonucleotides were dissociated from the target. These PP were amplified by PCR: the reaction was carried out in a total volume of 50 μl containing 0.1-100 fmols of the template in the presence of 1 μM each of the primers (FIG. 6), 100 mM of each dNTPs, 10 mM Tris-HCl, pH 8.3, 1.5 mM MgCl₂, 50 mM KCl, and 1 U of Platinum™ Taq polymerase (Invitrogen, CA). Typically, 27-30 PCR cycles were used, consisting each of 30 s at 94° C., 30 s at 53° C. and 30 s at 72° C. The quantity and quality of PCR products were estimated by agarose gel-electrophoresis and/or by measuring the 6-FAM fluorescence of PCR amplicons, after removal the non-incorporated primers using the Montage™ centrifuge filter device (Millipore, Billerica, Mass.). These products were rendered single stranded by incubation with 5 U of λ exonuclease (NEB, Boston, Mass.) that digests 5′-phosphorylated strand, for 30 min at 37° C., followed by 20 minutes at 65° C. to inactivate the enzyme. The same procedure was used to produce single stranded probes from PCR products from individual clones.

Hybridizations. The synthetic mixture of random oligonucleotides ROM22 (1 nmole) was used in the initial hybridization cycle to obtain the first affinity selected oligonucleotide mixture. In all subsequent hybridizations, the oligonucleotides obtained by affinity selection in the preceding cycle were PCR amplified and converted to the single stranded form. Typically, 10-50 pmoles of single stranded oligonucleotide mixture (0.05-0.25 μM) obtained in the previous cycle was mixed with two blocking oligonucleotides to obtain 0.5 μM each, in 200 ml of TMN buffer and heated to 90° C. This solution was subsequently transferred to tubes containing prebound biotinylated targets, then cooled down to the ambient temperature, 22-24° C., and left for at least 4 hours at this temperature. The tubes were then rinsed 3 times with TMN buffer and the probes that remained bound to the targets were washed off, by incubation at 90° C. in 200 ml of water, for 2 min. There was 1 pmole of the attached target per tube, except during the first hybridization when 100 pmoles were used. These hybridizations were followed by subtractive hybridizations carried as above but in the presence of 0.5 μM (total) of the non-desired oligonucleotide targets (i.e. other than the immobilized target).

Cloning and sequencing of individual probes. Cloning the probes from the affinity selected pooled probes was done using TOPO TA Cloning™ kit (Invitrogen, CA). Typically, ten positive clones were selected using X-Gal/IPTG based-colorimetric reaction, following the manufacturer's protocol. The M13 forward and reverse primers were used to confirm the presence of the insert and to “extract” it for subsequent direct sequence determination using LiCor apparatus (Lincoln, Nebr.). In turn, the cloned probes were produced by PCR using ROM22 primers and tested for binding. The cloned probe having signal/noise ratio bigger than 5 for all non-cognate targets was further analyzed. Typically it takes 1-2 clones to obtain such a signal/noise ratio.

### Results

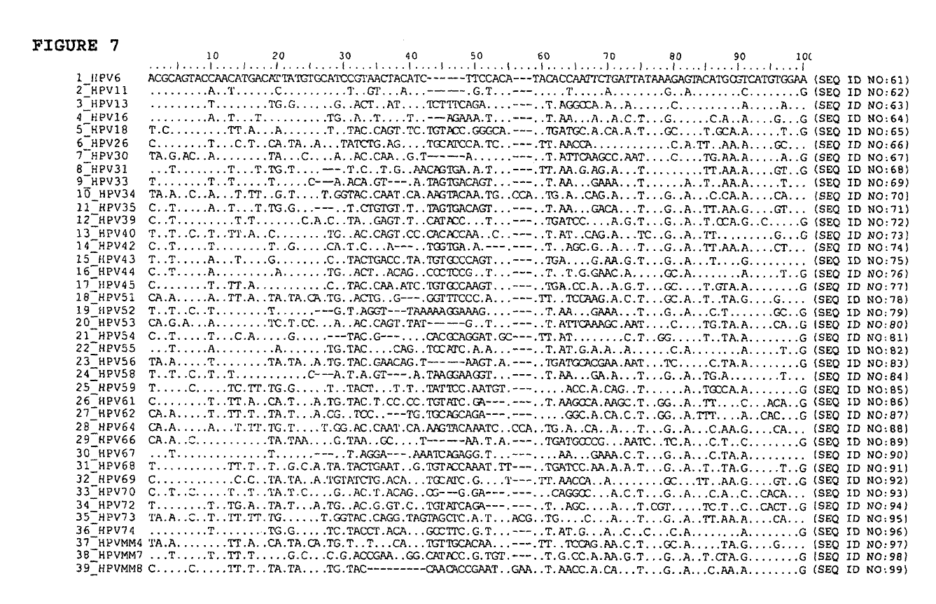

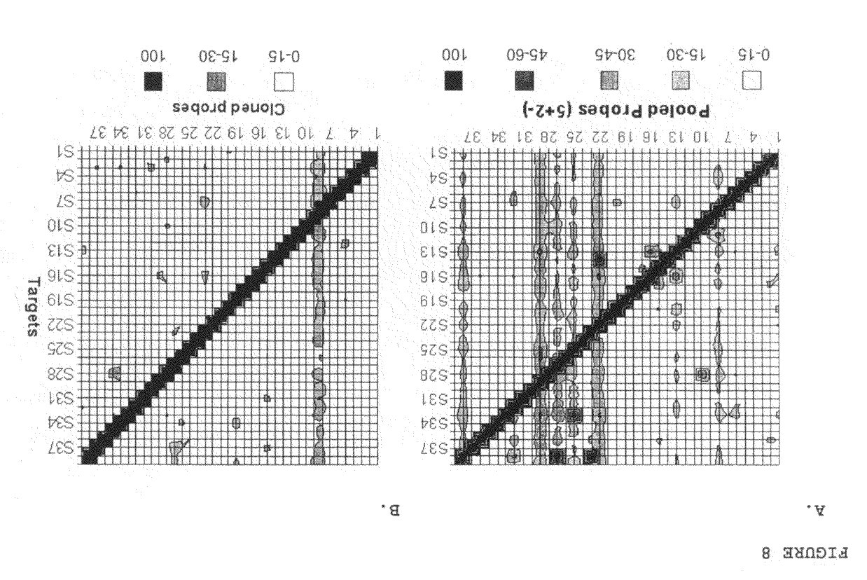

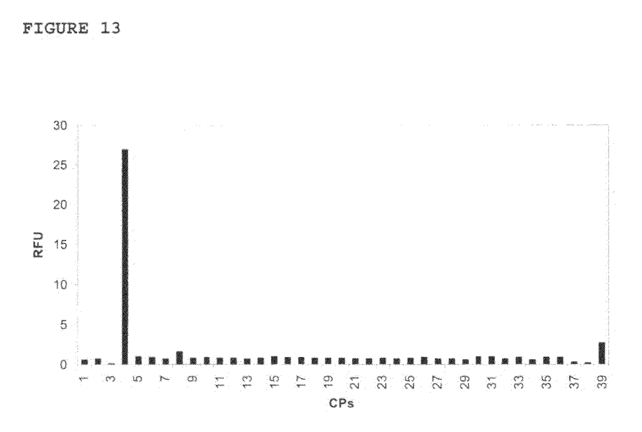

In the studies described herein, a series of iterative hybridizations were carried out to select probes recognizing 39 sequence variants of the “GP5+/6+” L1 region of HPV targets that are flanked by 20- and 23-nucleotide long “universal” primer sequences (FIG. 1). Targets, presenting different HPV types and consisting of 91-100 nucleotide-long oligonucleotides were chemically synthesized (IDT, Coralville, Iowa). The corresponding genomic segments (identical to targets) were aligned by ClustalW (Chema et al., (2003), supra) and presented in FIG. 7. The probes were obtained as described above. Briefly, biotinylated target oligonucleotides were immobilized in the streptavidin-coated tubes and hybridized to a mixture of synthetic random oligonucleotides, ROM22, consisting of a 22-nucleotide random sequence flanked by two 24-nucleotide long primer sequences. Following the first hybridization, the unbound ROM22 oligonucleotides were washed away and the bound ones were dissociated from their targets, re-amplified by PCR, and hybridized again. Each hybridization cycle enriched the resulting mixture of pooled probe sequences that efficiently binds to its target. The hybridization signal/noise ratio produced during hybridization was presented for each probe-target and probe-non-cognate target combination in the form of a matrix. As shown in FIG. 8A, the majority of pooled probes (PP) obtained after five iterative hybridizations and 2 cycles of subtractive hybridization (5+2−), bind to corresponding cognate target. In the next cycle we increased the stringency of subtractive hybridization, by increasing the concentration of particular non-cognate targets to the maximal level of 100 pmol per reaction. As shown in FIG. 8A, pooled probes that are specific for each of 39 HPV targets were obtained. FIG. 8B presents the data obtained following hybridizations of targets with cloned probes, which results in higher signal-to-noise ratios. These cloned probes are, on average, characterized by 10-fold stronger intensity of hybridization signal with cognate versus that of non-cognate targets. The 39 probes were simultaneously tested for each target under non-denaturing hybridization conditions and at room temperature thus confirming the robustness of the assay performance. FIG. 13 shows hybridization intensities of all selected type-specific CPs with the immobilized HPV16, the most common oncogenic HPV variant. The signal obtained with CP16 (CP #4 on the graph) was about 20 times stronger than with the remaining non-specific CPs.

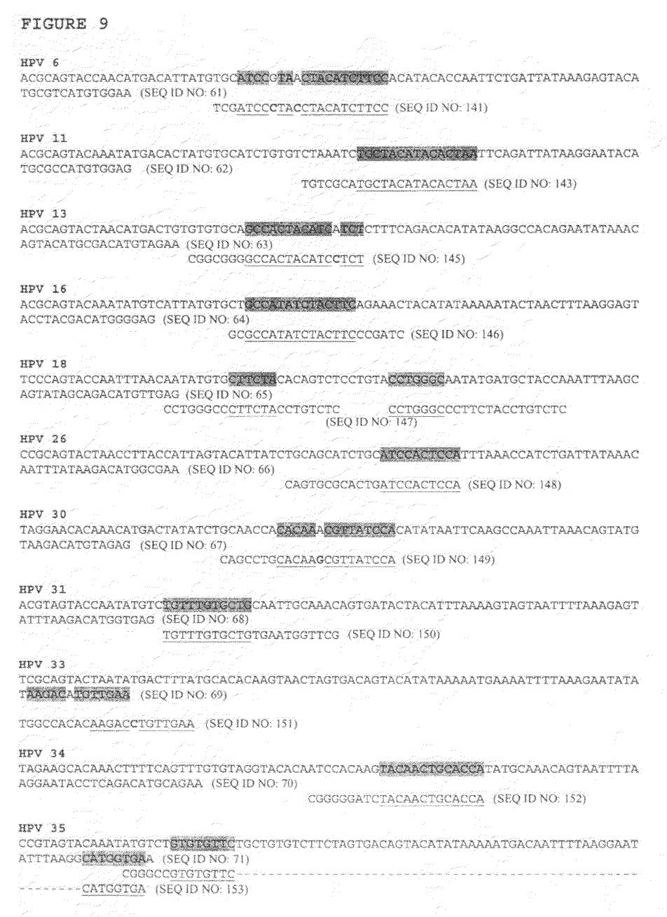

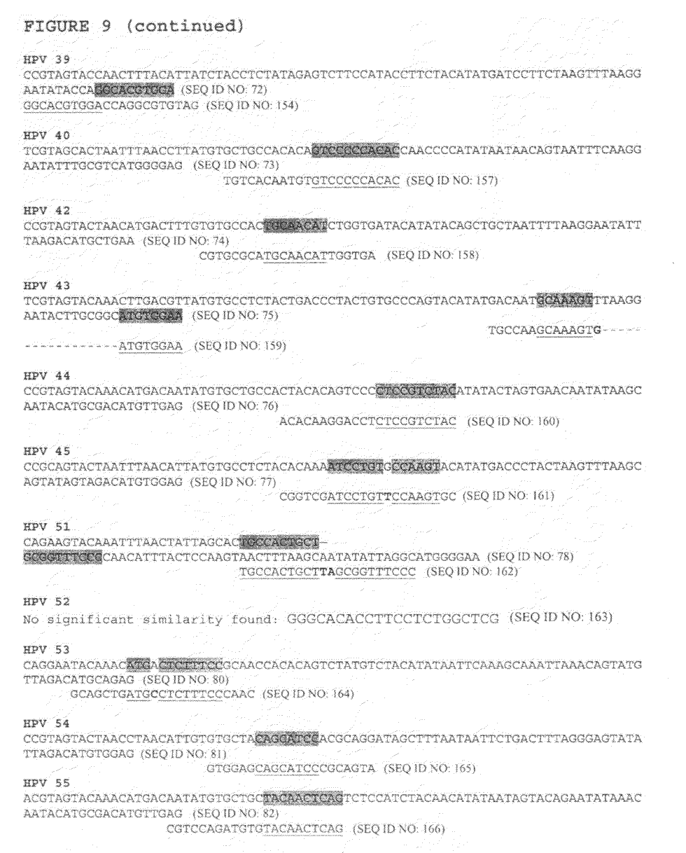

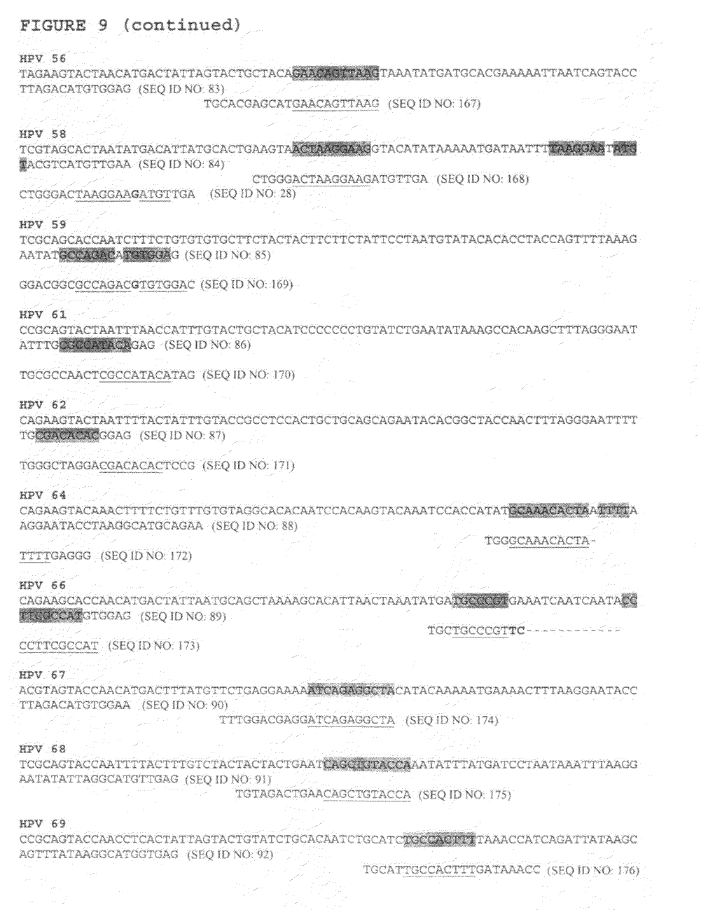

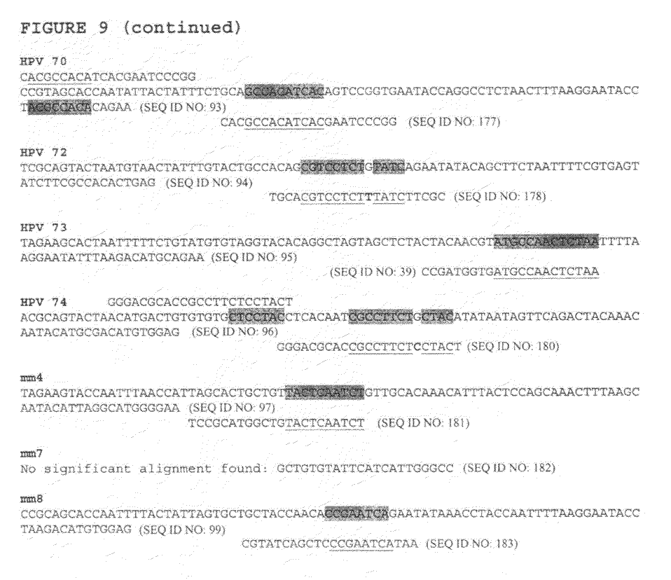

Each of the specific PPs, followed by 5+3− cycles of iterative hybridizations described above, consists of a mixture of different sequences. The corresponding unique sequence probes, CPs for cloned probes, were obtained by cloning PPs into plasmid vectors. Individual CPs were extracted from the obtained plasmids by PCR and tested for binding to the cognate and non-cognate targets. In 29 cases of type-specific PP, it took one clone to obtain desired signal/noise ratio of 10, or more. For ten PPs (type-specific for HPV 6, 34, 40, 43, 45, 52, 64, 70, 72 and MM7), all five tested clones continued to display 30%-50% cross-hybridization with 1 to 4 non-cognate targets. Therefore, in these cases, we performed additional subtractive hybridization (5+4−) using corresponding 5+3− PP and cross-hybridizing non-cognate targets. Clones of these PPs (5+4−) exhibited signal/noise ratios above a cut-off of 3.3. The CPs were sequenced and the reverse complement of some selected sequences are shown in FIG. 9.

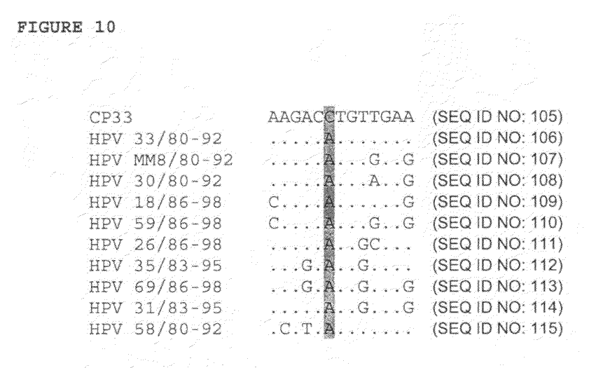

Obtained sequences allow examination of how this evolutionary approach beyond our rational design, generated best target-binders that at the same time do not bind to non-cognate targets (FIGS. 9 and 10).

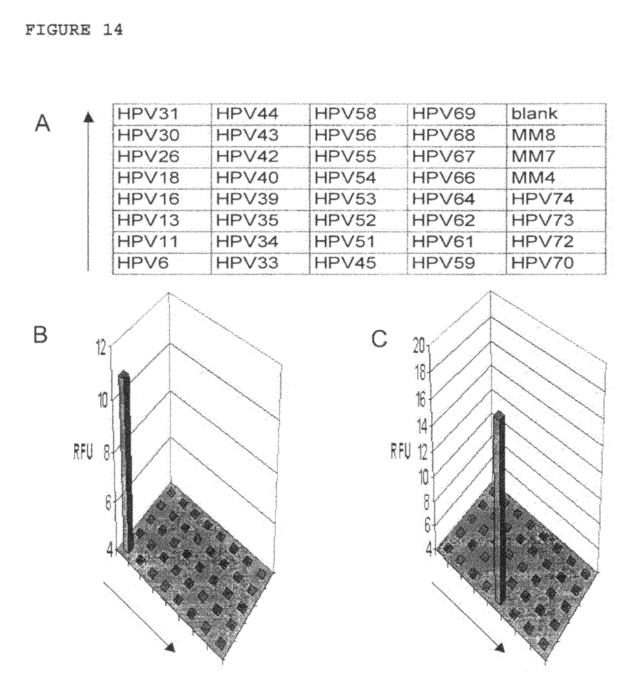

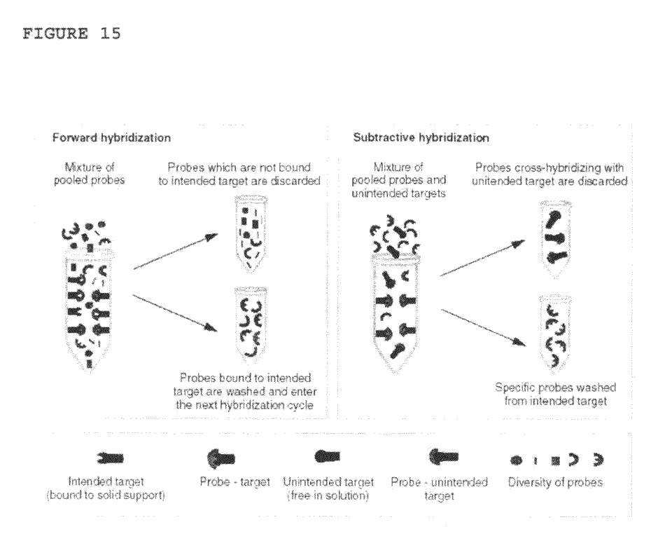

A HPV typing assay was performed in a reverse format, in which all 39 HPV type-specific CPs, biotinylated at the 5′ terminus were immobilized in streptavidin-coated plates (FIG. 14). Clinical samples containing HPV6 and HPV16 types were amplified by PCR using GP5+/6+ primers. The amplicons, converted to single stranded form, were hybridized to the panel of immobilized probes in the presence of the FAME-labelled detection probe and blocking oligonucleotides. As shown in FIGS. 14B and 14C, significant hybridization signal was only detected with CP6 and CP16.

Although the present invention has been described hereinabove by way of specific embodiments thereof, it can be modified, without departing from the spirit and nature of the subject invention as defined in the appended claims.

What is claimed is:

1. A method for identifying an oligonucleotide for discriminating a first nucleic acid from a second nucleic acid, said method comprising:
(a) hybridizing said first nucleic acid with a pool of oligonucleotides in a hybridization mixture, said oligonucleotides comprising a random nucleotide sequence flanked by primer recognition sequences;
(b) removing oligonucleotides which are not bound to said first nucleic acid from said hybridization mixture;
(c) dissociating bound oligonucleotides from said first nucleic acid;
(d) amplifying said bound oligonucleotides using primers capable of binding to said primer recognition sequences to obtain amplified oligonucleotide duplexes comprising a first strand corresponding to said bound oligonucleotides and a second strand corresponding to the complement of said bound oligonucleotides;
(e) treating said duplexes to remove or degrade said second strand to obtain single-stranded amplified oligonucleotides;
(f) repeating (a) to (e), wherein said pool of oligonucleotides of (a) is the amplified oligonucleotides obtained in (e) thereby to obtain further amplified oligonucleotides; and
(g) repeating (a) to (e), wherein said hybridization step (a) is performed in the further presence of said second nucleic acid;
wherein an oligonucleotide comprising the random nucleotide sequence of said further amplified oligonucleotides can be used for discriminating said first nucleic acid from said second nucleic acid.

2. The method according to claim 1, wherein said repeating step (f) is performed at least 2 times.

3. The method according to claim 2, wherein said repeating step (f) is performed at least 4 times.

4. The method according to any one of claims 1 to 3, wherein said repeating step (g) is performed at least 2 times.

5. The method according to claim 4, wherein said repeating step (g) is performed at least 3 times.

6. The method according to any one of claims 1 to 5, further comprising selecting an oligonucleotide from said further amplified oligonucleotides on the basis of its preferential binding to said first nucleic acid relative to said second nucleic target.

7. The method according to any one of claims 1 to 6, wherein said hybridization is performed in the presence of a blocking agent capable of inhibiting binding of said primer recognition sequences to said first nucleic acid.

8. The method according to claim 7 wherein said blocking agent is an oligonucleotide capable of binding said primer recognition sequences.

9. The method according to any one of claims 1-8, wherein said first nucleic acid is derived from a pathogen.

10. The method according to claim 9, wherein said pathogen is selected from a eukaryote, prokaryote and a virus.

11. The method according to claim 10, wherein said virus is human papillomavirus (HPV).

12. The method according to claim 11, wherein said first and second nucleic acids are derived from different subtypes of HPV.

13. The method according to claim 12, wherein said subtypes are selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

14. The method according to any one of claims 1-13, wherein said first nucleic acid is bound to a solid support.

15. The method according to claim 14, wherein the random nucleotide sequence of said further amplified oligonucleotides comprises at least 1 mismatch relative to said first nucleic acid.

16. The method according to any one of claims 1-15, wherein said amplification is performed using polymerase chain reaction (PCR) or isothermal amplification.

17. The method according to any one of claims 1-16, wherein said dissociation is performed by incubation at an elevated temperature relative to said hybridization.

18. The method according to claim 17, wherein said elevated temperature is a temperature above the thermal melting point (Tm).

19. The method according to any one of claims 1 to 18, wherein said treatment is with an exonuclease capable of selective degradation of said second strand.

20. An oligonucleotide identified by the method according to any one of claims 1-19.

21. An oligonucleotide capable of discriminating a first nucleic acid from a second nucleic acid, wherein said oligonucleotide is not exactly complementary to said first nucleic acid.

22. The oligonucleotide according to claim 21, wherein said oligonucleotide comprises at least 1 mismatch relative to said first nucleic acid.

23. The oligonucleotide according to claim 21 or 22, wherein said first nucleic acid is derived from a pathogen.

24. The oligonucleotide according to claim 23, wherein said pathogen is selected from a eukaryote, prokaryote and a virus.

25. The oligonucleotide according to claim 24, wherein said virus is human papillomavirus (HPV).

26. The oligonucleotide according to claim 25, wherein said first and second nucleic acids are derived from different subtypes of HPV.

27. The oligonucleotide according to claim 26, wherein said subtypes are selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

28. The oligonucleotide according to claim 27, comprising a nucleotide sequence selected from SEQ ID NOs: 1-43, 100-104 and 116.

29. The oligonucleotide according to claim 28, wherein said oligonucleotide comprises a sequence and is capable of selectively detecting an HPV subtype as set forth in Table I.

30. The probe according to claim 28 wherein said oligonucleotide is selected from:
an oligonucleotide comprising SEQ ID NO: 1, 2, 100 or 116 and which is capable of selectively detecting HPV 6;
an oligonucleotide comprising SEQ ID NO: 3, 4 or 101 and which is capable of selectively detecting HPV 11;
an oligonucleotide comprising SEQ ID NO: 5 and which is capable of selectively detecting HPV 13;
an oligonucleotide comprising SEQ ID NO: 6 or 102 and which is capable of selectively detecting HPV 16;
an oligonucleotide comprising SEQ ID NO: 7 or 103 and which is capable of selectively detecting HPV 18;
an oligonucleotide comprising SEQ ID NO: 8 and which is capable of selectively detecting HPV 26;
an oligonucleotide comprising SEQ ID NO: 9 and which is capable of selectively detecting HPV 30;
an oligonucleotide comprising SEQ ID NO: 10 and which is capable of selectively detecting HPV 31;
an oligonucleotide comprising SEQ ID NO: 11 and which is capable of selectively detecting HPV 33;
an oligonucleotide comprising SEQ ID NO: 12 and which is capable of selectively detecting HPV 34;
an oligonucleotide comprising SEQ ID NO: 13 and which is capable of selectively detecting HPV 39;
an oligonucleotide comprising SEQ ID NO: 14, 15 or 16 and which is capable of selectively detecting HPV 39;
an oligonucleotide comprising SEQ ID NO: 17 and which is capable of selectively detecting HPV 40;
an oligonucleotide comprising SEQ ID NO: 18 and which is capable of selectively detecting HPV 42;
an oligonucleotide comprising SEQ ID NO: 19 and which is capable of selectively detecting HPV 43;
an oligonucleotide comprising SEQ ID NO: 20 and which is capable of selectively detecting HPV 44;
an oligonucleotide comprising SEQ ID NO: 21 and which is capable of selectively detecting HPV 45;
an oligonucleotide comprising SEQ ID NO: 22 and which is capable of selectively detecting HPV 51;
an oligonucleotide comprising SEQ ID NO: 23 and which is capable of selectively detecting HPV 52;
an oligonucleotide comprising SEQ ID NO: 24 and which is capable of selectively detecting HPV 53;
an oligonucleotide comprising SEQ ID NO: 25 and which is capable of selectively detecting HPV 54;
an oligonucleotide comprising SEQ ID NO: 26 and which is capable of selectively detecting HPV 55;
an oligonucleotide comprising SEQ ID NO: 27 and which is capable of selectively detecting HPV 56;
an oligonucleotide comprising SEQ ID NO: 28 and which is capable of selectively detecting HPV 58;
an oligonucleotide comprising SEQ ID NO: 29 and which is capable of selectively detecting HPV 59;
an oligonucleotide comprising SEQ ID NO: 30 and which is capable of selectively detecting HPV 61;
an oligonucleotide comprising SEQ ID NO: 31 and which is capable of selectively detecting HPV 62;
an oligonucleotide comprising SEQ ID NO: 32 and which is capable of selectively detecting HPV 64;
an oligonucleotide comprising SEQ ID NO: 33 and which is capable of selectively detecting HPV 66;
an oligonucleotide comprising SEQ ID NO: 34 and which is capable of selectively detecting HPV 67;
an oligonucleotide comprising SEQ ID NO: 35 and which is capable of selectively detecting HPV 68;
an oligonucleotide comprising SEQ ID NO: 36 and which is capable of selectively detecting HPV 69;
an oligonucleotide comprising SEQ ID NO: 37 and which is capable of selectively detecting HPV 70;
an oligonucleotide comprising SEQ ID NO: 38 and which is capable of selectively detecting HPV 72;
an oligonucleotide comprising SEQ ID NO: 39 and which is capable of selectively detecting HPV 73;
an oligonucleotide comprising SEQ ID NO: 40 and which is capable of selectively detecting HPV 74;
an oligonucleotide comprising SEQ ID NO: 41 and which is capable of selectively detecting HPV MM4;
an oligonucleotide comprising SEQ ID NO: 42 and which is capable of selectively detecting HPV MM7;
an oligonucleotide comprising SEQ ID NO: 43 and which is capable of selectively detecting HPV MM8; and
an oligonucleotide comprising SEQ ID NO: 104 and which is capable of selectively detecting HPV 31 and/or 33.

31. An oligonucleotide comprising a nucleotide sequence selected from SEQ ID NOs: 1-43, 100-104 and 116.

32. A method for detecting the presence or absence of a first nucleic acid in a sample, said method comprising contacting the oligonucleotide according to any one of claims 20-31 with said sample under conditions permitting selective hybridization of said oligonucleotide to said first nucleic acid, wherein said selective hybridization is indicative that said first nucleic acid is present in said sample.

33. The method according to claim 32, wherein said first nucleic acid is derived from a pathogen and said method is for detection of said pathogen in a sample.

34. The method according to claim 33, wherein said sample is a biological sample derived from a subject and said method is for detection of said pathogen in said subject.

35. The method according to claim 34, wherein said method is for diagnosing a disease or condition associated with said pathogen in said subject.

36. The method according to any one of claims 33-35, wherein said pathogen is selected from a eukaryote, prokaryote and a virus.

37. The method according to claim 36, wherein said virus is human papillomavirus (HPV).

38. The method according to claim 37, wherein said method is for detecting the presence of a subtype of HPV.

39. The method according to claim 38, wherein said subtype is selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

40. The method according to claim 39, wherein said oligonucleotide is selected from:
an oligonucleotide comprising SEQ ID NO: 1, 2, 100 or 116 and which is capable of selectively detecting HPV 6;
an oligonucleotide comprising SEQ ID NO: 3, 4 or 101 and which is capable of selectively detecting HPV 11;
an oligonucleotide comprising SEQ ID NO: 5 and which is capable of selectively detecting HPV 13;
an oligonucleotide comprising SEQ ID NO: 6 or 102 and which is capable of selectively detecting HPV 16;
an oligonucleotide comprising SEQ ID NO: 7 or 103 and which is capable of selectively detecting HPV 18;
an oligonucleotide comprising SEQ ID NO: 8 and which is capable of selectively detecting HPV 26;
an oligonucleotide comprising SEQ ID NO: 9 and which is capable of selectively detecting HPV 30;
an oligonucleotide comprising SEQ ID NO: 10 and which is capable of selectively detecting HPV 31,
an oligonucleotide comprising SEQ ID NO: 11 and which is capable of selectively detecting HPV 33;
an oligonucleotide comprising SEQ ID NO: 12 and which is capable of selectively detecting HPV 34;
an oligonucleotide comprising SEQ ID NO: 13 and which is capable of selectively detecting HPV 39;
an oligonucleotide comprising SEQ ID NO: 14, 15 or 16 and which is capable of selectively detecting HPV 39;
an oligonucleotide comprising SEQ ID NO: 17 and which is capable of selectively detecting HPV 40;
an oligonucleotide comprising SEQ ID NO: 18 and which is capable of selectively detecting HPV 42;
an oligonucleotide comprising SEQ ID NO: 19 and which is capable of selectively detecting HPV 43;
an oligonucleotide comprising SEQ ID NO: 20 and which is capable of selectively detecting HPV 44;
an oligonucleotide comprising SEQ ID NO: 21 and which is capable of selectively detecting HPV 45;
an oligonucleotide comprising SEQ ID NO: 22 and which is capable of selectively detecting HPV 51;
an oligonucleotide comprising SEQ ID NO: 23 and which is capable of selectively detecting HPV 52;
an oligonucleotide comprising SEQ ID NO: 24 and which is capable of selectively detecting HPV 53;
an oligonucleotide comprising SEQ ID NO: 25 and which is capable of selectively detecting HPV 54;
an oligonucleotide comprising SEQ ID NO: 26 and which is capable of selectively detecting HPV 55;
an oligonucleotide comprising SEQ ID NO: 27 and which is capable of selectively detecting HPV 56;
an oligonucleotide comprising SEQ ID NO: 28 and which is capable of selectively detecting HPV 58;
an oligonucleotide comprising SEQ ID NO: 29 and which is capable of selectively detecting HPV 59;
an oligonucleotide comprising SEQ ID NO: 30 and which is capable of selectively detecting HPV 61;
an oligonucleotide comprising SEQ ID NO: 31 and which is capable of selectively detecting HPV 62;
an oligonucleotide comprising SEQ ID NO: 32 and which is capable of selectively detecting HPV 64;
an oligonucleotide comprising SEQ ID NO: 33 and which is capable of selectively detecting HPV 66;
an oligonucleotide comprising SEQ ID NO: 34 and which is capable of selectively detecting HPV 67;
an oligonucleotide comprising SEQ ID NO: 35 and which is capable of selectively detecting HPV 68;
an oligonucleotide comprising SEQ ID NO: 36 and which is capable of selectively detecting HPV 69;
an oligonucleotide comprising SEQ ID NO: 37 and which is capable of selectively detecting HPV 70;
an oligonucleotide comprising SEQ ID NO: 38 and which is capable of selectively detecting HPV 72;
an oligonucleotide comprising SEQ ID NO: 39 and which is capable of selectively detecting HPV 73;
an oligonucleotide comprising SEQ ID NO: 40 and which is capable of selectively detecting HPV 74;
an oligonucleotide comprising SEQ ID NO: 41 and which is capable of selectively detecting HPV MM4;
an oligonucleotide comprising SEQ ID NO: 42 and which is capable of selectively detecting HPV MM7;
an oligonucleotide comprising SEQ ID NO: 43 and which is capable of selectively detecting HPV MM8; and
an oligonucleotide comprising SEQ ID NO: 104 and which is capable of selectively detecting HPV 31 and/or 33.

41. The method according to claim 34 or 36, wherein said subject is a mammal.

42. The method according to claim 41, wherein said mammal is a human.

43. The method according to any one of claims 32-42, wherein said oligonucleotide is bound to a solid support.

44. The method according to any one of claims 32-43, wherein said first nucleic acid is labelled with a detectable marker.

45. The method according to claim 44, wherein said detectable marker is a fluorescent moiety.

46. A kit for detecting the presence of a first nucleic acid in a sample, said kit comprising the oligonucleotide according to any one of claims 20-31.

47. The kit of claim 46, further comprising means for detecting selective hybridization of said oligonucleotide to said first nucleic acid.

48. The kit according to claim 46 or 47, further comprising instructions setting forth the method of claim 31.

49. The kit according to any one of claims 46 to 48, wherein said first nucleic acid is derived from a pathogen and said kit is for detecting the presence of said pathogen in said sample.

50. The kit according to claim 49, wherein said sample is a biological sample derived from a subject and said kit is for detection of said pathogen in said subject.

51. The kit according to claim 50, wherein said kit is for diagnosing a disease or condition associated with said pathogen in said subject.

52. The kit according to any one of claims 48-51, wherein said pathogen is selected from a eukaryote, prokaryote and a virus.

53. The kit according to claim 52, wherein said virus is human papillomavirus (HPV).

54. The kit according to claim 53, wherein said kit is for detecting the presence of a subtype of HPV.

55. The kit according to claim 54, wherein said subtype is selected from HPV 6, HPV 11, HPV 13, HPV 16, HPV 18, HPV 26, HPV 30, HPV 31, HPV 33, HPV 34, HPV 35, HPV 39, HPV 40, HPV 42, HPV 43, HPV 44, HPV 45, HPV 51, HPV 52, HPV 53, HPV 54, HPV 55, HPV 56, HPV 58, HPV 59, HPV 61, HPV 62, HPV 64, HPV 66, HPV 67, HPV 68, HPV 69, HPV 70, HPV 72, HPV 73, HPV 74, HPV MM4, HPV MM7 and HPV MM8.

56. The kit according to any one of claims 46-55, wherein said oligonucleotide is the oligonucleotide of claim 30.

57. A collection of two or more oligonucleotides, wherein said oligonucleotides comprise a nucleotide sequence selected from the group consisting of SEQ ID NOs: 1-43, 100-104 and 116.

58. The collection according to claim 57, wherein said oligonucleotides are immobilized on a substrate.

59. The collection of any one of claims 57-58, wherein said oligonucleotides are hybridizable array elements in a microarray.

60. An array comprising the oligonucleotide according to any one of claims 20-31 or the collection of two or more oligonucleotides according to any one of claims 57-59.

61. A kit for identifying an oligonucleotide for discriminating a first nucleic acid from a second nucleic acid, said kit comprising the pool of oligonucleotides defined in any one of claims 1-19.

62. The kit according to claim 61, further comprising instructions setting forth the method according to any one of claims 1-19.
